(19) United States
(12) Patent Application Publication
(10) Pub. No.: US 2022/0287625 A1
(43) Pub. Date: Sep. 15, 2022

(54) SYSTEM AND METHOD FOR ADJUSTMENTS OF JOINTS AND SPINE AND MECHANICAL MOTION THERAPY

(71) Applicant: SIGMA INSTRUMENTS HOLDINGS, LLC, Cranberry Twp., PA (US)

(72) Inventors: JOHN CRUNICK, Cranberry Twp., PA (US); MAURICE PISCIOTTANO, Cranberry Twp., PA (US); LOUIS L. LASKEY, JR., Cranberry Twp., PA (US)

(73) Assignee: SIGMA INSTRUMENTS HOLDINGS, LLC, Cranberry Twp., PA (US)

(21) Appl. No.: 17/632,287

(22) PCT Filed: Aug. 3, 2020
(86) PCT No.: PCT/US20/44780

§ 371 Date: Feb. 2, 2022

Related U.S. Application Data
(60) Provisional application No. 62/882,274, filed on Aug. 2, 2019.

(51) Int. Cl. A61B 5/00; A61H 1/00

(52) U.S. Cl. CPC A61B 5/4528; A61B 5/6843; A61B 5/0051; A61H 1/006; A61N 1/06

(57) ABSTRACT

A system for treating a joint of a patient includes a treatment head including a probe, a force impulse wave sensor, and a pressure sensor. When the probe is pressed against the joint and reaches a predetermined pressure, the pressure sensor causes a release of current such that the probe delivers a mechanical force impulse to the joint. The force impulse wave sensor is configured to sense a frequency of the mechanical force impulse associated with a treatment point of the joint. The treatment head remains in the same target spot during the pre-test, treatment, and post-test. The system is configured to perform the pretest and post-test analysis of the plurality of treatment points to evaluate improvement of the joint after the treatment. The treatment protocol for the treatment point is modified based on the sensed frequency of the mechanical force impulse from the pre-test at the treatment point.

### CROSS-REFERENCE TO RELATED APPLICATION

This application claims the benefit of U.S. Provisional Application No. 62/882,274, filed Aug. 2, 2019, which is hereby incorporated by reference in its entirety.

### FIELD

The disclosure is directed to medical systems and methods. More specifically, the present disclosure relates to medical systems for, and methods of, treating joints, spine, and soft tissue of a patient in a medical environment such as, for example, physical therapy.

### BACKGROUND

Measurements and treatments of joints, the spine, and soft tissue have been an issue in manual medicine since its inception. Doctors and therapist have always relied on their skills to be able to assess and treat joints, the spine, and soft tissue problems. One problem is that it is difficult to accurately deliver and/or record the applied forces and scientifically measure the results via a dynamic response before, during, or after treatment.

It is with these thoughts in mind, among others, that aspects of the system and method for adjustments of joints and spine and mechanical motion therapy were developed.

### BRIEF SUMMARY

Aspects of the present disclosure may include a system for treating a joint of a patient. The system may include a treatment head and a computing device. The treatment head may include a probe tip, a pressure sensor, and a wave sensor. The treatment head may be configured to mechanically oscillate the probe tip so as to provide treatment. The computing device may be in communication with the treatment head and may include a processing device and a computer-readable medium with one or more executable instructions stored thereon. Wherein the processing device of the computing device executes the one or more instructions to perform the following operations. Receiving, from the wave sensor, a first pre-test waveform representing a first factor of joint mobility at a first location on the patient. Determining a first treatment protocol for use at the first location based on the first factor of joint mobility at the first location. Signaling the treatment head to mechanically oscillate the probe tip at the first location according to the first treatment protocol including a force, amplitude, and cycles. Receiving, from the wave sensor, a first post-test waveform representing a second factor of joint mobility at the first location on the patient. And determining a first measure of joint mobility based on a comparison of the first and second factors of joint mobility.

In certain instances, the operations further include the following steps. Receiving, from the wave sensor, a second pre-test waveform representing a third factor of joint mobility at a second location on the patient. Determining a second treatment protocol for use at the second location based on the third factor of joint mobility at the second location. Signaling the treatment head to mechanically oscillate the probe tip at the second location according to the second treatment protocol including a force, amplitude, and cycles. Receiving, from the wave sensor, a second post-test waveform representing a fourth factor of joint mobility at the second location on the patient. And determining a second measure of joint mobility based on a comparison of the third and fourth factors of joint mobility.

In certain instances, the first and second locations are on opposite sides of the joint. In certain instances, the first and second locations are on a posterior side of the joint and an anterior side of the joint, respectively. In certain instances, the first and second locations are on a superior side of the joint and an inferior side of the joint, respectively.

In certain instances, the operations further include calculating a joint function index based on the first and second measures of joint mobility. In certain instances, calculating the joint function index includes a calculation of the differences between the first pre-test waveform and the first post-test waveform and the difference between the second pre-test waveform and the second post-test waveform. In certain instances, calculating the joint function index includes a summation of: a first difference between the first pre-test waveform and the first post-test waveform; and a second difference between the second pre-test waveform and the second post-test waveform.

In certain instances, the treatment head includes an electrode operably coupled to the probe, the electrode being configured to deliver an electrical stimulation to the patient

In an embodiment, a system is provided for treating a joint of a patient. The system includes a treatment head including a probe, a force impulse wave sensor, and a pressure sensor. The pressure sensor is configured so that when the probe is pressed against the joint and reaches a predetermined pressure, the pressure sensor causes a release of current such that the probe delivers a mechanical force impulse to the joint. The force impulse wave sensor is configured to sense a frequency of the mechanical force impulse associated with at least one treatment point of the joint. The system may also include a computer portion including a central processing unit (CPU) in communication with the treatment head. The system may further include a first display screen in communication with the computer portion. The first display screen is configured to display a plurality of treatment points on the joint for pre-test, treatment, and post-test for placing the treatment head, to display the pre-test and post test results of each target spot. The treatment head remains in the same target spot during the pre-test, treatment, and post-test. The computer portion is configured to perform the pretest and post-test analysis of the plurality of treatment points to evaluate improvement of the joint after the treatment. At least one treatment protocol for the at least treatment point is modified based on the sensed frequency of the mechanical force impulse from the pre-test at the at least one treatment point.

In some embodiments, the treatment may include a number of impulses or impacts of a force at a frequency.

In some embodiments, the frequency ranges from 0.1 Hz to 12 Hz.

In some embodiments, the joint may include one of shoulder, elbow, wrist, hip, knee, or ankle.

In some embodiments, the plurality of treatment points for the shoulder or hip is at least 5. The plurality of treatment points for the wrist is at least 3. The plurality of treatment points for the ankle is at least 4.

In some embodiments, the computer portion further includes a memory. The computer portion causes the electrical stimulation to be delivered relative to the mechanical force impulse according to the at least one treatment protocol.

In some embodiments, the treatment protocol causes the electrical stimulation to be delivered subsequent to the delivery of the mechanical force impulse.

In some embodiments, the system may also include a second display screen in communication with the computer portion, the second display screen configured to display a highlighted target area for treatment on a live human model and to display an application of the treatment head on the highlighted treatment point of the live human model.

In some embodiments, the system calculates a joint function compliance based upon the pre-tests and post-tests on the plurality of treatment points.

In some embodiments, the joint function compliance reflects the improvement of the joint mobility after the treatment of the joint.

In some embodiments, the system may also include an electrode operably coupled to the probe to deliver an electrical stimulation.

In an embodiment, a system is provided for treating soft tissue of a patient. The system may include a treatment head including a probe, and a pressure sensor configured so that when the probe is pressed against the soft tissue and reaches a predetermined pressure, the pressure sensor causes a release of current such that the probe delivers a mechanical force impulse to the soft tissue. The system may also include a memory device in communication with the treatment head, the memory device storing a plurality of treatment protocols for a plurality of treatment points on the soft tissue of the patient. The system may also include a computer portion including a central processing unit (CPU) in communication with the memory device, and a display screen in in communication with the computer portion. The display screen is configured to display the plurality of treatment points and select at least one of the plurality of treatment points of the soft tissue for placing the treatment head and to show the patient how to move the patient's body during the treatment of the soft tissue. The computer portion is configured to perform the treatment of the at least one of the plurality of treatment points according to the at least one of the plurality of treatment protocols when the patient performs one or more motions near the at least one of the plurality of treatment points.

In some embodiments, the soft tissue may include one of the plurality of treatment points comprising shoulder, elbow, wrist, hand, hips, knee, ankle, feet, neck, lower back, middle back, pelvis, ribs, arm, and leg.

In some embodiments, the treatment head treats the soft tissue at a frequency ranging from 12 Hz to 30 Hz.

In some embodiments, the treatment head is configured to treat the soft tissue to create one or more neuro-pathways in the soft tissue.

In some embodiments, the system may also include an electrode operably coupled to the probe to deliver an electrical stimulation.

In an embodiment, a system is provided for treating a spine of a patient. The system may include a treatment head including a probe, a force impulse wave sensor, and a pressure sensor. The pressure sensor is configured so that when the probe is pressed against the spine and reaches a predetermined pressure, the pressure sensor causes a release of current such that the probe delivers a mechanical force impulse to the spine. The force impulse wave sensor is configured to sense a frequency of the mechanical force impulse associated with at least one treatment point of the spine. The system may also include a computer portion including a central processing unit (CPU) in communication with the treatment head. The system may further include a first display screen in communication with the computer portion. The first display screen is configured to display a plurality of treatment points on the spine for pre-test, treatment, and post-test with the treatment head. At least one treatment protocol for the at least treatment point is modified based on the sensed frequencies of the mechanical force impulses at least two or more of the treatment points. The computer portion is configured to perform the pretest analysis and post-test analysis of the plurality of treatment points to evaluate the improvement of the spine after the treatment.

In some embodiments, the spine may include one of cervical spine portion, thoracic spine portion, lumbar spine portion, or sacral spine portion.

In some embodiments, the cervical spine portion may include seven treatment points C1-C7 for treatment.

In some embodiments, the thoracic spine portion may include eleven treatment points T1-T11 for treatment.

In some embodiments, the lumbar spine portion may include five treatment points L1-L5 for treatment.

In some embodiments, the sacral spine portion may include five treatment points S1-S for treatment.

In some embodiments, the system may also include a second display screen in communication with the computer portion, the second display screen configured to display a highlighted target area for treatment on a live human model and to display an application of the treatment head on the highlighted treatment point of the live human model.

In some embodiments, the system is configured to select one of a plurality of sub-harmonic ranges of the resonant frequency for the treatment of the spine.

In some embodiments, the system may also include an electrode operably coupled to the probe to deliver an electrical stimulation.

In an embodiment, a method of treating joint of a patient is provided. The method may include a) contacting the patient at a first location of a plurality of locations at least partially surrounding the joint with a probe tip of a treatment head of a treatment system. The treatment head may include a probe having the probe tip, a pressure sensor, and a wave sensor and is configured to mechanically oscillate the probe tip so as to provide treatment. The treatment head is in communication with a computer of the treatment system. The computer may include a memory and a central processing unit (CPU). The computer is in communication with a display device of the treatment system. The method may also include b) applying a first compressive force with the probe tip to the first location proximate the joint, thereby the wave sensor receives a pre-test waveform representing a first factor of joint mobility at the first location. The CPU may determine a first treatment protocol for use at the first location based on the first factor of joint mobility at the first location. The method may further include c) applying a second compressive force with the probe tip at the first location, the second compressive force causing the probe tip of the treatment head to deliver percussive impacts according to the first treatment protocol including a force, amplitude, and cycles. The method may also include d) applying a third compressive force with the probe tip at the first location, thereby the wave sensor receives a post-test waveform representing a second factor of joint mobility at the first location. The CPU may determine a first measure of joint mobility based on a comparison of the first and second factors of joint mobility.

In some embodiments, the method may also include repeating the steps of (a)-(d) to each of a second location, a third location, a fourth location, and a fifth location.

In some embodiments, the first location is on a posterior side of the joint, the second location is on an anterior side of the joint, the third location is on a lateral side of the joint, the fourth location is on a superior side of the joint, and the fifth location is on an inferior side of the joint.

In some embodiments, the computer portion calculates a joint function index based on the pre-tests of the five locations.

In some embodiments, the percussive impacts are delivered to the first location.

In some embodiments, the percussive impacts and an electrical stimulation delivered to the first location are delivered generally simultaneously. The electrical stimulation is created from an electrode operably coupled to the probe.

In some embodiments, the joint may include one of shoulder, elbow, wrist, hip, knee, or ankle.

In an embodiment, a method of treating spine of a patient is provided. The method may include a) contacting the patient at a first location of a plurality of locations at least partially surrounding the spine with a probe tip of a treatment head of a treatment system. The treatment head may include a probe having the probe tip, a pressure sensor, and a wave sensor. The treatment head is configured to mechanically oscillate the probe tip so as to provide treatment and is in communication with a computer of the treatment system. The computer may include a memory and a central processing unit (CPU), the computer in communication with a display device of the treatment system. The method may also include b) applying a first compressive force with the probe tip to the first location proximate the spine. The method may further include c) repeating the steps of a) and b) for each of the remaining plurality of locations, thereby the wave sensor receives a pre-test waveform representing a first factor of spine mobility at the plurality of locations. The method may also include d) selecting a treatment location from the plurality of locations. The CPU may determine a treatment protocol for use at the treatment location based on the first factor of spine mobility at the treatment location and at least one of the first factor of spine mobility at one or more of the plurality of locations. The method may also include e) applying a second compressive force with the probe tip at the treatment location, the second compressive force causing the probe tip of the treatment head to deliver percussive impacts according to the treatment protocol including a force, amplitude, and cycles. The method may further include f) applying a third compressive force with the probe tip at the plurality of locations, thereby the wave sensor receives a post-test waveform representing a second factor of spine mobility at the plurality of locations. The CPU may determine a measure of spine mobility based on a comparison of the first and second factors of spine mobility.

In some embodiments, the percussive impacts are delivered to the treatment location.

In some embodiments, the spine may include one of cervical spine portion, thoracic spine portion, lumbar spine portion, or sacral spine portion.

In some embodiments, each of cervical spine portion, thoracic spine portion, lumbar spine portion, and sacral spine portion may include a respective plurality of treatment points.

In an embodiment, a method of treating a soft tissue of a patient is provided. The method may include a) contacting the patient at a target treatment location of the soft tissue with a probe tip of a treatment head of a treatment system. The treatment head may include a probe having the probe tip and a pressure sensor, and is configured to mechanically oscillate the probe tip so as to provide treatment. The treatment head is in communication with a computer of the treatment system. The computer may include a memory and a central processing unit (CPU). The memory may store a plurality of pre-determined treatment protocols. The computer is in communication with a display device of the treatment system. The treatment head may contact the patient at a target treatment location. The method may also include b) applying a preload tissue compression force to the target treatment location and use a pressure sensor to determine the preload tissue compression force, the pressure sensor being configured so that when the probe is pressed against the tissue and reaches a predetermined pressure, the pressure sensor causes a release of current such that the probe delivers a mechanical force impulse to the soft tissue. The method may also include c) delivering percussive impacts to the target treatment location based upon at least one of the plurality of pre-determined treatment protocols when the patient performs one or more motions near the target treatment location.

In some embodiments, the percussive impacts and an electrical stimulation delivered to the target treatment location are delivered generally simultaneously. The electrical stimulation is created from an electrode operably coupled to the probe.

In some embodiments, the soft tissue may include one of the plurality of treatment points comprising shoulder, elbow, wrist, hand, hips, knee, ankle, feet, neck, lower back, middle back, pelvis, ribs, arm, and leg.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure, wherein:

FIG. 1 is a cross-sectional side view of an impulse and sensing head of the system in accordance with an embodiment of the disclosure;

FIG. 2 is a schematic diagram showing the hardware components of the system used to create and capture the wave form in accordance with an embodiment of the disclosure;

FIG. 3 depicts the thoracic analysis computer screen in accordance with an embodiment of the disclosure;

FIG. 4 depicts the lateral cervical analysis computer screen in accordance with an embodiment of the disclosure;

FIG. 5 shows a computer screen depicting a wave form which has derived information from each of the screens shown in FIGS. 3 and 4.

FIGS. 6A-J are diagrams of embodiments of probes for an impulse stimulator instrument;

FIG. 7 is a schematic diagram illustrating a module for joint analysis in accordance with an embodiment of the disclosure;

FIG. 8 is a schematic diagram illustrating improvement analysis after treatment for a joint in accordance with an embodiment of the disclosure;

FIG. 9 is a flow chart illustrating the steps of performing the treatment for the joint in accordance with an embodiment of the disclosure;

FIG. 10 is an embodiment of the system displaying a home screen;

FIG. 11A is an embodiment of a patient-education screen displaying particular joint regions on one side;

FIG. 11B is an embodiment of a measurement and analysis screen on another side;

FIG. 12 is an embodiment of the patient-education screen displaying that shoulder is selected;

FIG. 13 is an embodiment of the measurement and analysis screen displaying the five spots for shoulder;

FIG. 14 is an embodiment of the measurement and analysis screen displaying the five spots for elbow;

FIG. 15 is an embodiment of the measurement and analysis screen displaying the five spots for wrist;

FIG. 16 is an embodiment of the measurement and analysis screen displaying the five spots for knee;

FIG. 17 is an embodiment of the measurement and analysis screen displaying the five spots for hip;

FIG. 18 is an embodiment of the measurement and analysis screen displaying the five spots for ankle;

FIG. 19 is an embodiment of the measurement and analysis screen displaying the first spot selected for shoulder and the force, frequency, the limits of impacts, mode, and preload;

FIG. 20 is an embodiment of the measurement and analysis screen displaying treatment conditions including force, frequency, and limits of impacts, mode, and preload and the joint function compliance for shoulder;

FIG. 21A is an embodiment of a display including two display screens for the joint analysis;

FIG. 21B is a flow chart illustrating the educational module for the joint treatment;

FIG. 22 is a flow chart illustrating the steps of performing the treatment for the joint in accordance with an embodiment of the disclosure;

FIG. 23 is a schematic diagram illustrating a spine module for spine treatment and analysis in accordance with an embodiment of the disclosure;

FIG. 24 is a flow chart illustrating the educational module for the spine treatment in accordance with an embodiment of the disclosure;

FIG. 25 is a diagram illustrating a full spine displaying treatment spots in accordance with an embodiment of the disclosure;

FIG. 26 is an embodiment of the patient-education screen displaying particular spine regions after the spine button of FIG. 10 is touched;

FIG. 27A is an embodiment of a display including two display screens for the spine analysis;

FIG. 27B is a flow chart illustrating the steps of displaying on the patient-education screen for the spine in accordance with an embodiment of the disclosure;

FIG. 28A illustrates the soft tissue treatment head with a higher frequency in accordance with an embodiment of the disclosure;

FIG. 28B illustrates the joint and spine treatment head with a lower frequency in accordance with an embodiment of the disclosure;

FIG. 28C illustrates a treatment head without a piezoelectric sensor in the treatment of the soft tissue of FIG. 28A in accordance with an embodiment of the disclosure;

FIG. 28D illustrates a treatment head including a piezoelectric sensor in the treatment of the joint and spine of FIG. 28B in accordance with an embodiment of the disclosure;

FIG. 29 is a schematic diagram illustrating a soft tissue module for the soft tissue treatment in accordance with an embodiment of the disclosure;

FIG. 30 is a flow chart illustrating the steps of performing the treatment for the soft tissue in accordance with an embodiment of the disclosure;

FIG. 31 is an embodiment of a home screen displaying sixteen buttons for treatment of soft tissue;

FIG. 32 is an embodiment of a screen displaying four protocols after shoulder button on the home screen of FIG. 31 is touched; and

FIG. 33 is an embodiment of a screen displaying an animated motion of video following the screen of FIG. 32.

### DETAILED DESCRIPTION

The disclosure may be understood by reference to the following detailed description, taken in conjunction with the drawings as described below. It is noted that, for purposes of illustrative clarity, certain elements in various drawings may not be drawn to scale.

### I. System Overview

Disclosed herein is a system 1111 for, and method of, measuring and treating joints and/or the spine of a patient, or treating the soft tissue. The system 1111 is configured for imparting a repetitive mechanical force into the spine, joint, or soft tissue. The system 1111 may also be configured to provide electrical stimulation via electrodes 14. The system 1111 may also be configured to record via a computer program 38 the results of the imparted force and/or the electrical stimulation.

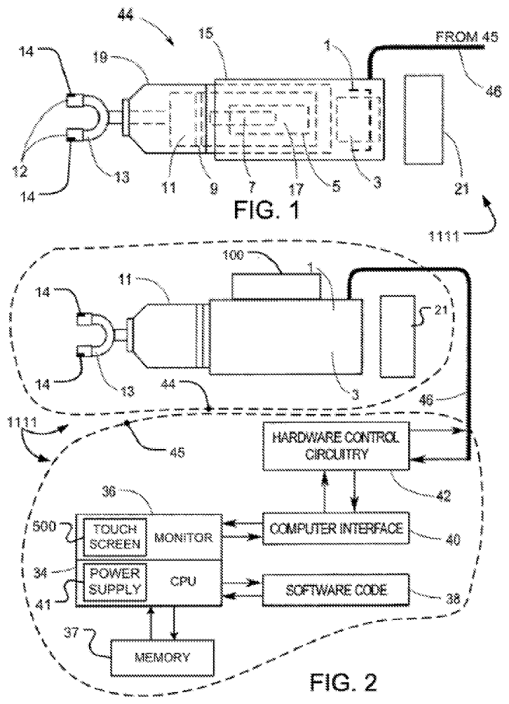

In one embodiment, as seen in FIGS. 1 and 2, the system 1111 is configured for the measurement of spine and joint response arising from the application of a force impulse. In one embodiment, the system 1111 includes an impulse and sensing head 44 (also referred to as a “treatment head” or “treatment device”) capable of determining tissue response. The impulse and sensing head 44 is configured to apply a percussive force impulse to spine and joint in an oscillating fashion. The head 44 may be used on the joints of the body including, for example, the shoulder, elbow, wrist, hip, knee, and ankle, or a combination thereof. In response to an initial or test force applied to the patient's body, a wave form characteristic of the energy absorption profile is generated and transmitted back to the impulse and sensing head 44. The system 1111 may use the wave form to develop custom treatment plans for the patient for the particular area of the body that was tested. Additionally, the impulse and sensing head 44 may include conductive probes 13 for the purpose of providing electrical stimulation, which is computer controlled, to the skin and dermatomes.

The system 1111, for example, at its impulse and sensing head 44, includes signal generating components attached to the data acquisition circuitry 45 of the head 44 so a signal will be captured by the data acquisition circuitry of the computer portion 45 of the system 1111. Data acquisition circuitry of the computer portion 45 also captures the wave form and a signal characteristic of the resultant force impulse that is indicative of the energy absorption of said tissue.

In one embodiment, as seen in FIGS. 1 and 2, the impulse and sensing head 44 includes a probe 13, a piezoelectric sensor 11 firmly attached to the probe 13, an anvil 9 firmly attached to the sensor 11, an electromagnetic coil 5 and an armature 7. The armature 7 is inserted without attachment into the electromagnetic coil 5 and configured so that when the coil 5 is energized, the armature 7 is accelerated to impact the anvil 9 and thereby produce the force impulse, which travels through the piezoelectric sensor 11 and causes the piezoelectric sensor 11 to generate the wave form. A pressure sensor 3 is attached to the head 44 and configured so that when the probe 13 is pressed against the tissue and reaches a predetermined pressure, the pressure sensor 3 causes a release of a burst of current that energizes the electromagnetic coil 5. The pressure sensor 3 is also attached to the signal generating components, which output data, characteristic of the pressure of the probe 13 in contact with the tissue, to the computer 45.

In one embodiment, the tip of the impulse and sensing head 44 is constructed with electrodes 14 that are designed to make contact with the skin. At the same instant the force impulse is delivered via the armature 7 being accelerated to impact the anvil 9, an electric pulse is generated and delivered via electrodes 14 to the patient in either a continuous current or as a pulse as selected within the software 38. Features of the system 1111 including the impulse and sensing head 44 may include features of the system and device shown and described in U.S. Pat. No. 10,226,397, filed Dec. 9, 2016, U.S. Pat. No. 9,782,324, filed Mar. 11, 2014, and/or U.S. Patent Publication 2015/0080990, filed Sep. 24, 2014. All of these Patents and Applications are hereby incorporated by reference in their entireties into the present application for all purposes.

In one embodiment, the data acquisition circuitry 45 includes a computer 34 connected to a display screen 36. An illustration of the joint, spine, and/or soft tissue may be displayed on the screen 36. Information indicating the force impulse, the pressure of the probe 13 and the wave form may be stored in the computer 34. This information can be merged together, sorted, and logged for each patient. The computer 34 can recall and print this information. The software 38 also allows for various configurations of the electrical stimulation impulse that allows for various types of waveforms and frequencies and power settings.

The graphic display on the computer screen 36 is configured to show parts of the body and allows the doctor or therapist to choose the area of the measurement by using a touch screen 500 to identify and log the area of measurement. Additionally there are pre-programmed protocols that can be used to guide the doctor in the application of the system 1111 for specific conditions.

The system 1111 uses a computer algorithm that may use baseline muscle tension data and/or baseline ligament tension data to give the doctor or therapist information regarding the characteristics of the joint or spine.

The system 1111 can also be used to treat patients. The probe 13 of the impulse and sensing head 44 may oscillate by repetitively accelerating the armature 7 to impact the anvil 9 at a controlled frequency and a predetermined time period. In certain instances, electrodes 14 on the tips 12 of the probes 13 may be used to administer electrical stimulation at the tips 12 of the probes 13. In some variations, the system 1111 can be applied to the joint, spine, or soft tissue to reset the firing patterns of muscle spindle fibers via force impulses. In some variations, the system 1111 can be applied to the joint, spine, or soft tissue to reset the firing patterns of muscle spindle fibers via force impulses while at the same time exciting muscle spindle fibers and dermatomes with electrical stimulation.

In some variations, the frequency may be varied between approximately 0.1 Hertz and approximately 30 Hertz in increments of approximately 0.1 Hertz. The electrical stimulation falls within the range used for this common therapy. For example, the electrical stimulation may be varied between approximately 0.1 and approximately 150 Hz.

In some variations, the frequency ranges from 0.1 Hz to 12 Hz for treating the spine or joint. In some variations, the frequency is equal to or greater than 0.1 Hz for treating the spine or joint. In some variations, the frequency is equal to or greater than 1 Hz for treating the spine or joint. In some variations, the frequency is equal to or greater than 2 Hz for treating the spine or joint. In some variations, the frequency is equal to or greater than 4 Hz for treating the spine or joint. In some variations, the frequency is equal to or greater than 6 Hz for treating the spine or joint. In some variations, the frequency is equal to or greater than 8 Hz for treating the spine or joint. In some variations, the frequency is equal to or greater than 10 Hz for treating the spine or joint. In some variations, the frequency is equal to or less than 12 Hz for treating the spine or joint. In some variations, the frequency is equal to or less than 10 Hz for treating the spine or joint. In some variations, the frequency is equal to or less than 8 Hz for treating the spine or joint. In some variations, the frequency is equal to or less than 6 Hz for treating the spine or joint. In some variations, the frequency is equal to or less than 4 Hz for treating the spine or joint. In some variations, the frequency is equal to or less than 2 Hz for treating the spine or joint. In some variations, the frequency is equal to or less than 1 Hz for treating the spine or joint. In some variations, the frequency ranges from 12 Hz to 30 Hz for treating soft tissue.

In some variations, the frequency is equal to or greater than 12 Hz for treating the soft tissue. In some variations, the frequency is equal to or greater than 14 Hz for treating the soft tissue. In some variations, the frequency is equal to or greater than 16 Hz for treating the soft tissue. In some variations, the frequency is equal to or greater than 18 Hz for treating the soft tissue. In some variations, the frequency is equal to or greater than 20 Hz for treating the soft tissue. In some variations, the frequency is equal to or greater than 22 Hz for treating the soft tissue. In some variations, the frequency is equal to or greater than 24 Hz for treating the soft tissue. In some variations, the frequency is equal to or greater than 26 Hz for treating the soft tissue. In some variations, the frequency is equal to or greater than 28 Hz for treating the soft tissue. In some variations, the frequency is equal to or less than 30 Hz for treating the soft tissue. In some variations, the frequency is equal to or less than 28 Hz for treating the soft tissue. In some variations, the frequency is equal to or less than 26 Hz for treating the soft tissue. In some variations, the frequency is equal to or less than 24 Hz for treating the soft tissue. In some variations, the frequency is equal to or less than 22 Hz for treating the soft tissue. In some variations, the frequency is equal to or less than 20 Hz for treating the soft tissue. In some variations, the frequency is equal to or less than 18 Hz for treating the soft tissue. In some variations, the frequency is equal to or less than 16 Hz for treating the soft tissue. In some variations, the frequency is equal to or less than 14 Hz for treating the soft tissue. With respect to spine or joint measurement via piezoelectric sensing devices 11 and the logging of the amplitude of the wave form output from such piezoelectric sensing devices 11, there is complexity in the differing shapes of the wave forms elicited during the mobility testing of spine or joint. Initial experiments and demonstrations have shown that there is useful information trapped in each wave form output of a piezoelectric sensor 11 interposed in a percussion system for testing spine or joint response. The system 1111 employs a method of capturing the mathematic representations of the wave form output from the percussive testing of spine or joint and then manipulating and interpreting such mathematic representations so as to define the amount of spine or joint mobility and the condition and characteristics of such spine or joint mobility.

The system 1111 is configured to analyze the relationship of all of the response factors associated with spine or joint measurement and treatment, namely the analysis of the waveforms as they relate to spine or joint in general. The relation to the stiffness characteristic (waveform peak), the hysteresis function (wave shape), and the frequency response provide valuable information regarding the state of the measured tissue.

In one embodiment, the electrical stimulation unit 100 of the system 1111 employs a high frequency oscillator 105 and a power amplifier 110 to generate a high frequency electrical signal that is then delivered to a transducer, such as an electrode 14. The electrical energy is then transmitted to the patient by applying a probe contact supported electrode against the patient's skin. The amplitude of the electrical signal plays a role in the electrical stimulation of the system 1111 because the lower the amplitude of the electrical signal, the more tolerant the patient is to the stimulation transmitted by the electrode 14.

All tissues in the human body, including skin, have the ability to conduct electricity. Indeed, this is how nerves function to relay information from one part of the body to another. The skin also has electrical activity, which is in constant, slight variation, and can be measured and charted. The skin's electrical conductivity fluctuates based on certain bodily conditions, and this fluctuation is called the galvanic skin response.

Sudden changes in emotion, such as fright, can trigger the galvanic skin response, as can other types of changes, such as the hot flashes that are characteristic of menopause. The galvanic skin response can be graphed on a chart for observation, in the same way that heart or brain activity is recorded.

In one embodiment of the system 1111, the galvanic response of the soft tissue being treated is measured via a conductive sensor 14 to calculate a change in the galvanic response being brought about by the treatment. This change in galvanic response of the soft tissue being treated is used to determine if, and how, the electrical stimulation of the treatment should be changed.

In one embodiment of the system 111, the system 111 includes electrical control circuitry 300 that includes a high frequency oscillator and a power amplifier to generate a high frequency electrical signal that is then delivered to a transducer, such as an electrode 14. The electrical energy is then transmitted to the patient by applying a probe 13 containing the electrode 14 against the patient's skin. The amplitude of the electrical signal is of interest in these electrical stimulation systems because the lower the amplitude of the electrical signal, the more tolerant the patient is to the stimulation transmitted by the electrode 14.

In one embodiment, the electrical stimulation involves placing the electrode 14 on the skin and using various waveforms to stimulate a tissue response, such as, for example, a muscle response in a passive manner.

In one embodiment, the system 1111 will apply a pre load response to compress the tissues during treatment. Pacinian corpuscles are pressure receptors located in the skin and also in various internal organs. Each Pacinian corpuscle is connected to a sensory neuron. When pressure is applied via the system probe 13, the pressure receptors elicit a response. However, the pressure receptors adapt very quickly and therefore stop firing. With the system 1111, the pressure that is applied via the probe 13 is augmented by the electrical stimulation provided via the electrodes 14 so as to deter the adaptation and increase the firing rate of the neural channel in addition to the electrical stimulation.

In one embodiment, the system 1111 will also produce during treatment a pressure wave that will stimulate motor neurons (e.g., type I-A) to activate a stretch reflex response. Other areas of the nervous system, such as, for example, nerve roots and ganglia, are also considered targets for this therapy capable of being delivered via the system 1111.

### II. Impulse and Sensing Head of the System

To begin a more detailed discussion of the features, components and operation of the system 1111, reference is made to FIG. 1, which is a cross-sectional side view of an impulse and sensing head 44. As shown in FIG. 1, the system 1111 for measurement of spine or joint mobility may be portable and hand-held and may include a delivery head 44 with an elongated generally cylindrical housing 15 which has an insert 19 that tapers to form a generally conical configuration at the forward end 20. The other end of the housing 15 is provided with a cylindrical closed end 21. The housing 15 and the closed end 21 may be separately connected by a screw threaded connection to provide access into the interior of the housing 15 and to separate the components of the disclosure for repair, replacement and the like. After housing 15 is unscrewed from closed end 21, it can slide back and insert 19 can also be unscrewed from the housing 15.

A probe 13 is located at the forward end 20 of the housing 15 and includes cushioned tips 12 for contacting the joint or spine to be measured. The probe 13 may be constructed of a rigid material such as metal, plastic, or the like. The probe 13 screws into or frictionally inserts into the piezoelectric sensor 11. Different shaped probes 13 may be used depending on if the apparatus is being used to improve joint mobility of an extremity or the spine, or if the apparatus is being used for therapeutic purposes on soft tissue.

In some variations, electrodes 14 may be supported on the probe 13, for example, at the cushioned tips 12, such that the electrodes 14 make good electrical contact with the soft tissue when the probe is applied to the patient.

Within the housing 15 is a solenoid assembly 17. The assembly 17 includes an electromagnetic coil 5 and an armature 7 longitudinally reciprocally mounted without attachment within the coil 5. The armature 7 is configured so that the end of the armature 7 will impact against the anvil 9 when the electromagnetic coil 5 is energized. The anvil 9 is affixed to one side of a piezoelectric sensor 11. The impact produces a force impulse which travels through the piezoelectric sensor 11 and causes the piezoelectric sensor 11 to generate a wave form. When any one of the various probes is placed against the joint or spine of a patient, the other end of the probe 13 resides firmly against the piezoelectric sensor 11 which in turn resides firmly against the anvil 9. A pressure sensor 3 that resides within the housing 15 is interposed between the closed end 21 of the housing 15 and the solenoid 17. The pressure sensor 3, works in concert with each of the other components so that upon reaching a point that corresponds to a predetermined pressure against the joint, spine, or soft tissue of a human subject, the pressure sensor 3 causes the release of a burst of current that energizes the electromagnetic coil 5 such that the armature 7 is accelerated to impact with the anvil 9. This action of the pressure sensor 3 may be linked with specific actions associated with a particular treatment protocol. More specifically, the impulse and sensing head 44 may perform differently when the pressure sensor 3 senses the predetermined pressure depending on the progress within a particular treatment protocol. For example, given a particular treatment protocol, the first time the pressure sensor 3 senses a predetermined pressure, the head 44 may generate a single force impulse to cause the probe tip 12 to contact the patient's tissue, and the head 44 may record a pre-treatment waveform representative of the patient's tissue prior to treatment (as will be described subsequently). The waveform may contribute to the determination of treatment parameters of the treatment protocol. Next, a second time the pressure sensor 3 senses the predetermined pressure, the head 44 may perform a percussive, oscillating force impulse to the patient according to the treatment protocol determined specifically for the patient at the particular point of the pre-test. Finally, a third time the pressure sensor 3 senses the predetermined pressure, the head 44 may generate a single force impulse to cause the probe tip 12 to contact the patient's tissue, and the head 44 may record a post-treatment waveform representative of the patient's tissue after treatment. Thus, the impulse and sensing head 44 may perform different functions (e.g., pre-test, treatment, post-test); and each of the functions may be triggered to begin by the application of a compressive force of the probe tip of the impulse and sensing head 44 against the patient's tissue.

The pressure sensor 3 may be comprised of a load cell. The impact of said armature 7 against the anvil 9 produces a force impulse which travels directionally, in a continuum with the direction of the armature 7 at impact, through the piezoelectric sensor 11 while at the same time being influenced by the resistance placed upon the piezoelectric sensor 11 by the probe 13 which is contact with the patient. The kinetic energy at the point of impact causes the piezoelectric sensor 11 to emit an electronic wave form which is characteristic of all of the elements of the electromechanical system on one side of the sensor opposed by all of the human elements on the other side of the sensor. The wave form is captured by data acquisition circuitry within a computer portion 45 of the system 1111 and retained therein for wave form analysis by the application of certain algorithms. In some variations, the power supply 41 is in the computer portion 45 of the system 1111 or even in the CPU 34. An insulated cable 46 connects the delivery head 44 to computer portion 45 of the system 1111 and the power supply 41. Alternatively, the current may be supplied through an electrical cord that may be plugged into a suitable electrical outlet or the like which extends into the housing 15.

The mass of the armature 7 is substantially equal to the mass of the anvil 9 so that when the armature 7 strikes the anvil 9 it transfers the energy of the armature 7 to the patient through the cushioned probe 13. The initial positions of the coil and the probe 13 are fixed so that the energy of the system can only be varied by varying velocity of the armature 7 at the point of impact with the anvil 9. The velocity of the armature 7 can be varied by varying the force with which it is accelerated into the electromagnetic coil 5 which is proportional to the current flowing into the coils of the solenoid 17 which in turn is proportional to the voltage. The triggering point at which the solenoid 17 is actuated can be varied by the relative movement pressure of the housing 15 inwardly in relation to the solenoid 17 and the probe 13 so that when the preset pressure has been matched, an electrical circuit is completed to the electromagnetic coil 5.

A single multi-axis inclinometer, disposed within the head 44, will sense the angle of incidence of the probe 13 in contact with the joint or spine being tested simultaneously with the formation of the wave form. The inclinometer 1 is connected by hard-wiring or telemetry to the data acquisition circuitry of the computer portion 45 of the system 1111. A signal corresponding to the angle of incidence will be captured by the data acquisition circuitry of the computer portion 45 and retained for display on the computer screen 36.

As indicated in FIG. 1, the system may include an electrical stimulation unit 100, which employs a high frequency oscillator 105 and a power amplifier 110 to generate a high frequency electrical signal that is then delivered to a transducer, such as an electrode 14 electrically coupled to the electrical stimulation unit 100. The electrical energy is then transmitted to the patient by applying a probe contact supported electrode 14 against the patient's skin. In one embodiment, the electrical stimulation unit 100 is subject to a control sequence or software that causes the delivery of a continuous current or pulse current via the electrodes 14 to the soft tissue at generally the same instant the force impulse is delivered to the soft tissue via the probe 13.

In the one embodiment, the system 1111 herein described may be used for therapeutic as well as analytical applications. For example, after an analysis is completed, a health care practitioner may use oscillating percussion for treatment of joint or spine. This may be accomplished by repetitively accelerating the said armature 7 to impact the anvil 9 thereby causing the probe 13 to oscillate back and forth in a rhythmic fashion. The percussive force of the probe 13 may be applied to a soft tissue for the purpose of improving/reducing muscle spasm and/or resetting the firing pattern of the muscle spindle fiber as well as exciting neural pathways. This may be done at a controlled impulse frequency of repetitive force impulses at a predetermined time period or a time period selected by the computer as a result of software algorithms. In an embodiment, the frequency of percussion is varied between 4 and 12 Hertz in increments of 0.1 Hertz. Because there is an inclinometer 1 within the therapy delivery head 44, precise angles of therapy may be applied to the patient and documented for future reference. X-ray imaging or other medical imaging may also be used in conjunction with the system 1111 herein described for accurate estimation of the angle of incidence for therapeutic purposes.

The treatment of the soft tissue provided by the oscillating percussion treatment may be enhanced by the simultaneous delivery of electricity to the soft tissue. For example, the electricity may be caused to be administered continuously to the soft tissue over the course of the oscillating percussion treatment. Alternatively, the electricity may be caused to be administered to the soft tissue intermittently in such a manner that the electricity delivery is pulsed to coincide with each pulse of the oscillating percussion treatment. Alternatively, the electricity may be caused to be administered to the soft tissue intermittently in such a manner that the electricity delivery is pulsed to generally occur between the pulses of the oscillating percussion treatment. Also, the electricity may be administered before or after the percussive treatment.

Data characteristic of the angle of incidence, pressure of the probe 13 on the patient, the force impulse via the probe 13, is permanently stored in computer memory 37 for each area of joint or spine tested, inclusive of all of the tests performed on a given patient during a given session so that such information may be combined with the test interpretation as derived from the analysis of the elicited wave form for each joint or spine region tested. A basis or “base line” is provided for comparison to the test angle of incidence so that those test angles can be matched during the performance of additional testing. The stored angle of incidence information along with the test data analysis for each patient session can be recalled and printed. Any part or, if practical, all of the test history of any patient can be combined for inclusion on one or more computer media so as to enable transfer of the records to any other practitioner so equipped to use the information in the furtherance of the care of the patient. Because the test angle is recorded and permanently stored, another doctor giving a second opinion can use the same angle for testing. Therefore, the results of tests performed by different doctors will be more uniform.

FIG. 2 is a block diagram of the architecture of the computer portion 45 and piezoelectric impulse and sensing head 44 that form the system 1111. In one embodiment, the computer portion 45 of the system includes a CPU 34, a monitor 36, memory 37, software code 38, a computer interface 40 and hardware control circuitry 42. The electromechanical impulse and sensing head 44 is activated and controlled with the computer software code 38 written onto the CPU 34 that communicates through the interface 40 to hardware control circuitry 42 and to the impulse and sensing head 44. Signals from the sensors 11 within the impulse and sensing head 44 travel to the hardware control circuitry 42 for conditioning and transmittal through the computer interface 40 circuitry to the CPU 34. Software code 38 is used to control and direct all signals between the electromechanical component 44 and the computer portion 45. All relevant information generated by the processes of the system 1111 and used for the processes of the system 1111 are stored in a memory 37 in communication with the CPU 34. The relevant information may be recalled onto the monitor 36 or printed as required.

Similar to the electromechanical impulse and sensing head 44, the electrodes 14 are energized and controlled with computer software code 38 written onto the CPU 34 that communicates through the interface 40 to hardware control circuitry 42 and to the electrodes 14 and the electrical stimulation unit 100. Signals from the sensors 11 within the impulse and sensing head 44, from the electrodes 14 and/or from the components of the electrical stimulation unit 100 travel to the hardware control circuitry 42 for conditioning and transmittal through the computer interface 40 circuitry to the CPU 34. Software code 38 is used to control and direct all such signals between the aforementioned components of the delivery head 44 and the computer portion 45 of the system 1111. All relevant information generated by the process is stored and may be recalled onto the monitor 36 or printed as required.

The resulting wave form is sinusoidal and will be influenced by such things as tissue mobility or resistance to mobility, fascia tension, muscle tonicity, connective tissue resiliency or inertia, local edema, and etc. Each such wave form may be characterized mathematically by logging the peak amplitude, peak time, rise time, fall time, and slew rate. The mathematic values of the data logged will facilitate the calculation of frequency response and certain ratios that will mathematically define the wave form characteristics. By analyzing the mathematics of the wave form characteristics, certain assumptions can be made as to the functional characteristics of the tissue condition.

As the data are collected and logged and after all of the pertinent mathematic calculations are made, a graphic display of the wave form may be presented on a display device, such as, e.g., a computer monitor 36. In addition to the graphic display, the pertinent data and derived ratios may be displayed for assessment by the user of the equipment. The user will be one trained in the interpretation of the wave form shape and interpretation of the logged and derived mathematic information. The graphic displays plus all of the mathematic information as a result of joint or spine percussion testing and/or electrical stimulation may be stored and recalled whenever deemed necessary. As the data base grows and expands, clinical assumptions will yield to statistically valid probabilities and predictive diagnoses. A permanent record of each test of each patient may be stored and recalled as necessary. It may also be copied to electronic storage media, such as, for example, a computer thumb drive, so that it can be transferred to another computer.

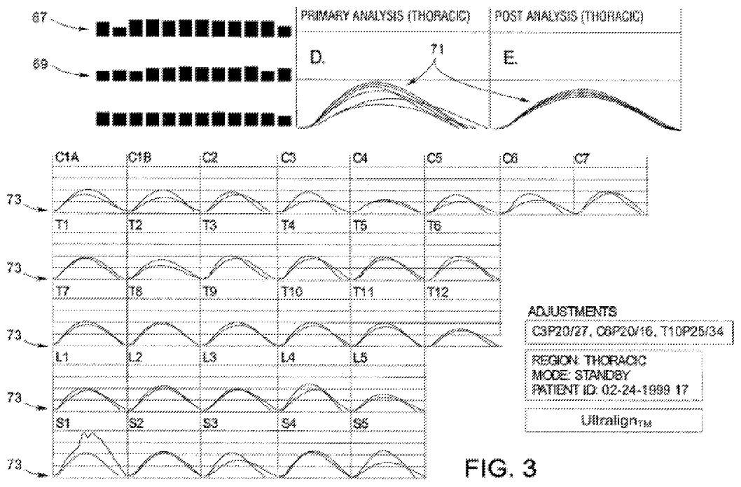

As each wave form is recovered from the piezoelectric sensor 11, several things become apparent. The amplitude of the wave form is of interest because as joint or spine mobility increases, the test wave form amplitude decreases. Therefore, in FIG. 3 a simple bar chart 67 is used for the expression of wave form peak amplitude. A statistical analysis (mean and standard deviation) of the amplitudes is included. Standard deviation may be set at one, two or three sigma and is expressed by a horizontal line on bar chart 69. The shape of the wave is an interesting piece of information. The expression of a ½ wave form 71 in a graphic display of the wave form shape for all joint or spine regions. A composite of all 7 Cervical, 12 Thoracic, or 10 Lumbosacral wave forms 73 is expressed before treatment and after treatment. A joint function compliance or index is shown for the joint.

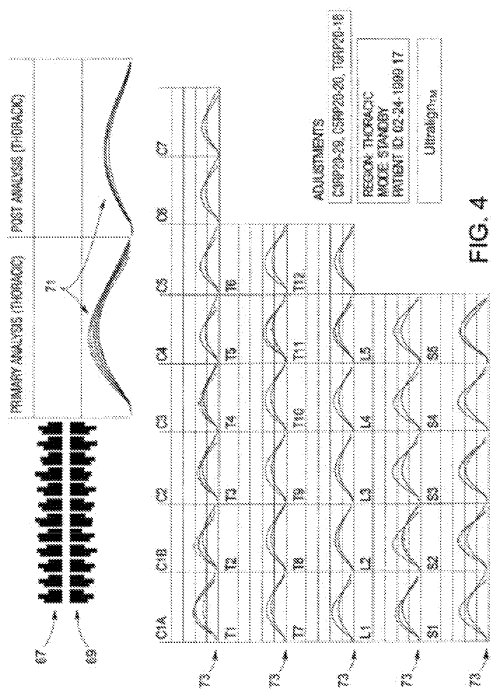

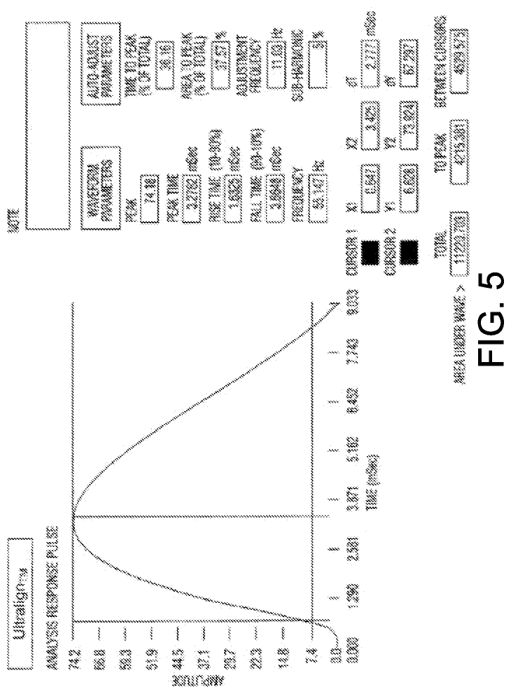

Each of the wave forms represented on FIG. 3 and FIG. 4 are analyzed for Peak Amplitude, Peak Time, Rise Time, Fall Time, Frequency (Hertz), Time (%) to Peak and Area (%) to Peak. The derived information is displayed as shown on FIG. 5 along with some calculated factors that are also shown. Any of the waveforms described herein may be analyzed for the factors shown in FIG. 5, such as peak, peak time, rise time, fall time, frequency, among other factors.

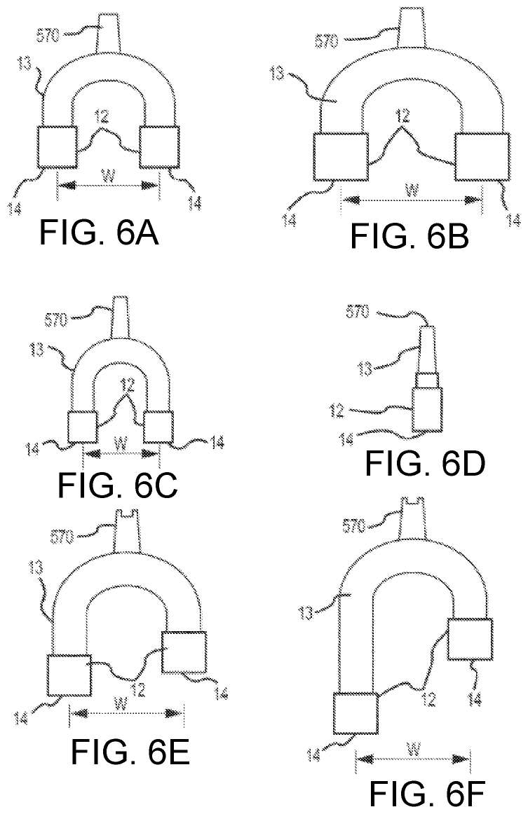

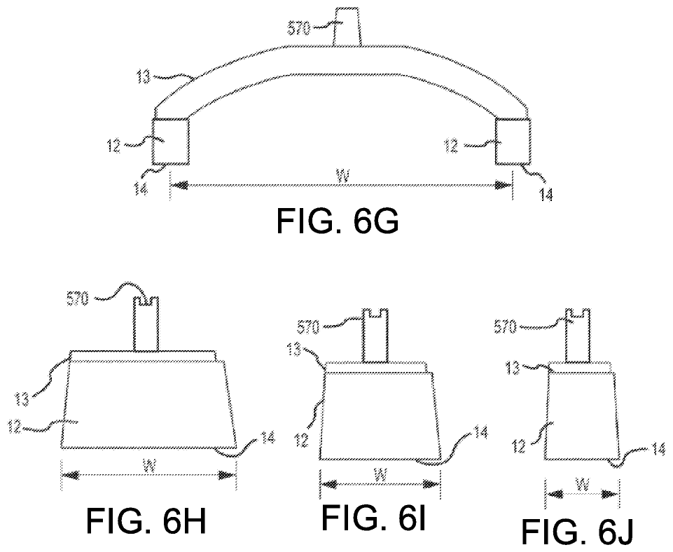

FIGS. 6A-6J are diagrams of embodiments of probes for an impulse stimulator instrument. As can be understood from FIGS. 6A-6J, a variety of different configurations of probes 13 can be employed with the therapy delivery head 44 of FIGS. 1 and 2. For example, as illustrated in FIGS. 6A-6C and 6E-6G, the probe 13 can have a generally horseshoe-shaped body ending is two space-apart tips 12, which may be soft. A stem 570 extends from the opposite side of the body of the probe 13 from the tips 12, the stem 570 being used for coupling the probe 13 to the forward end 20 of the head 44 and the piezoelectric sensor 11 and anvil 9, as can be understood from FIG. 1. Each tip 12 may have an electrode 14 at the extreme end of the tip 12.

As indicated in FIGS. 6A-6C and 6G, some dual tipped probes 13 may have tips 12 that extend generally an even distance. As shown in FIGS. 6E and 6F, other dual tipped probes 13 may have tips 12 that do not extend an even distance.

As can be understood from FIGS. 6A-6C and 6E-6G, the dual tipped probes 13 may have tips 12 that are laterally spaced apart from each other a variety of distances W. For example, the dual tipped probes 13 of FIGS. 6A-6C and 6E-6G have respective tip spacing distances W of 3.2 cm, 4.7 cm, 2.2 cm, 3.2 cm, 3.2 cm and 9.8 cm. As can be understood from a comparison of FIGS. 6E and 6F, despite having the same spacing distances W of 3.2 cm, the dual tipped probe 13 of FIG. 6F has a greater difference in the extent of extension of its tips 12 relative to each other than is the case with the tips 12 of the probe of FIG. 6E.

As can be understood from FIGS. 6D and 6H-6J, some embodiments of the probe 13 may have a single tip 12. A stem 570 extends from the opposite side of the body of the probe 13 from the tip 12, the stem 570 being used for coupling the probe 13 to the forward end 20 of the head 44 and the piezoelectric sensor 11 and anvil 9, as can be understood from FIG. 1. The tip 12 may have an optional electrode 14 at the extreme end of the tip 12.

In some cases, as in FIG. 6D, the single tip 12 may be generally hemispherical in configuration. In other cases, as in FIGS. 6H-6J, the single tip 12 may be generally flat-ended in configuration.

As can be understood from FIGS. 6H-6J, the flat-ended tips 12 may have a variety of widths W. For example, the flat-ended tips 12 depicted in FIGS. 6H-6J have respective widths W of 4.5 cm, 3.2 cm, and 1.7 cm. Such flat-ended tips 12 may be formed of soft rubber.

In some instances, the probes 13 of FIGS. 6A-6D may be employed where the patient tissue that is the target of the treatment being provide via the system 1111 is adjacent the patient's vertebra. The probes 13 of FIGS. 6E-6G may be employed on specific anatomical features of the patient. The probes 13 of FIGS. 6H-6J may be employed where the tissue being treated is in close proximity to skeletal structures.

In some variations, the electrode 14 is not present in the treatment head. In some variations, the electrode 14 is present in the treatment head.

More details about the system are disclosed in U.S. Pat. No. 10,226,397, entitled “System and Method For Treating Soft Tissue with Force Impulse and Electrical Stimulation,” by Tamas Becse et al, issued on Mar. 12, 2019, and in US Patent Publication 2015/0080990, entitled “System and Method for Treating Animals,” by John Crunick et al, published in on Mar. 18, 2015 (to be issued), each of which is incorporated herein by reference in its entirety for all purposes.

### III. Treatment Protocols and Methods for Improving Joint Mobility

One goal of the treatment protocols and methods described in this section is to improve or increase joint mobility or fluid mechanical motion of the joint. The treatment to the joint may include releasing and lining up the bones of the joint in a proper and healthy orientation.

As described in the section II. of this application, an exemplary treatment protocol may include a pre-treatment test of the joint mobility, a treatment to the joint, and post-treatment test of the joint mobility, all using the treatment head 44 of FIG. 1. In certain instances, a particular treatment protocol may involve testing and treating (e.g., pre-treatment test, treatment, post-treatment test) one or more points associated with a particular joint, and determining a joint function index or a measure of the improvement of the joint mobility. The measure of the improvement of the joint mobility may indicate that further treatment is necessary or not. For instances, if the joint function index indicated that there was little or no change from the pre-test as compared to the post-test, then subsequent treatment would be needed to further improve mobility of the joint.

During the pre-treatment and post-treatment test of a given treatment protocol, the treatment head 44 may receive a resulting waveform from the patient's tissue in response to a force impact from the treatment head 44 against the patient's joint. The piezoelectric sensor of the treatment head 44 may receive and record the resonant frequency of the patient's tissue at the particular point of testing. A treatment plan may be determined based on the resonant frequency of the resultant waveform. For example, a subharmonic frequency of the resonant frequency may be used, along with a particular intensity (force), amplitude of the impulse, number of cycles of the impulse, etc. The treatment head 44 may use a joint tip such as shown in FIG. 6H for the joint treatment and analysis. The system can perform pre-test, pre-test analysis, treatment, post-test, and post-test analysis.

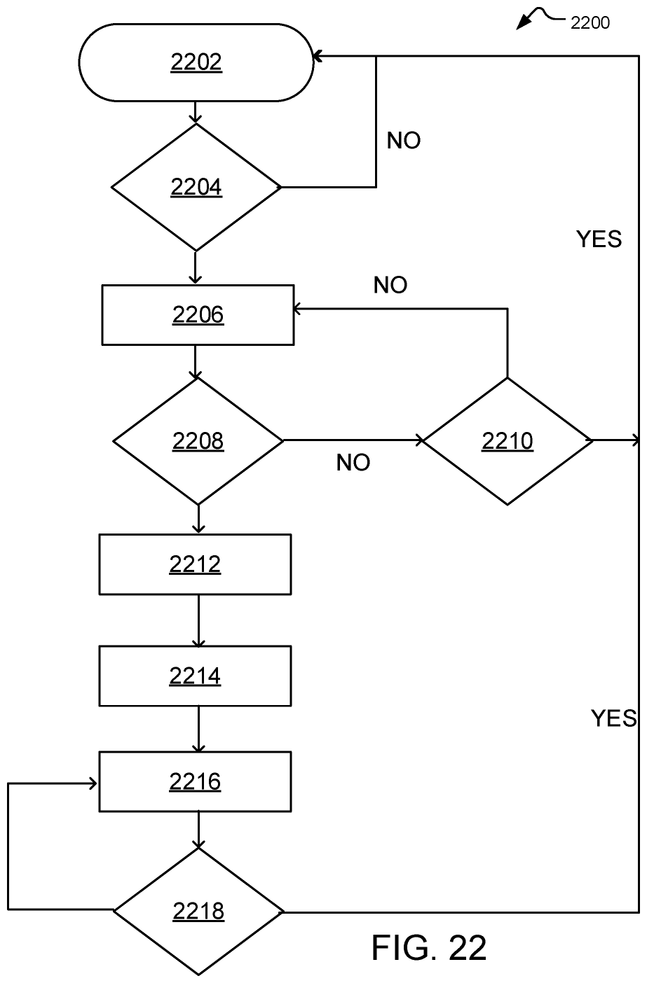

Reference is made to FIG. 22, which shows a flow chart illustrating exemplary steps of applying the pre-test in order to determine treatment parameters for treatment of a joint. A method 2200 for the system to operate may include reading head sensor (e.g. pressure sensor 3) at step 2202. The method 2200 may also include checking if the initial threshold is met at 2204. If not, the system goes back to read the pressure sensor 3 again until the initial threshold is met.

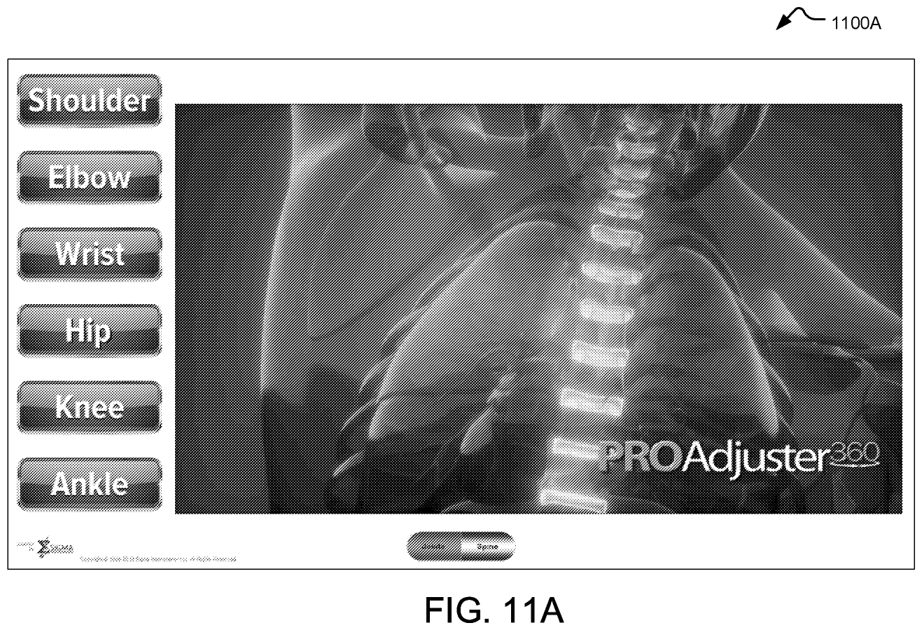

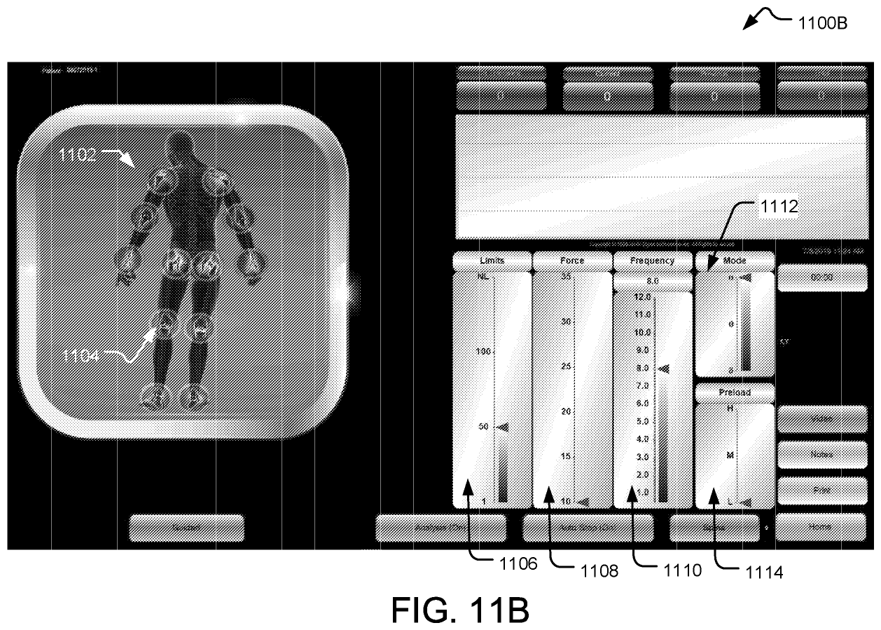

The method 2200 continues with acquiring sensor reading (e.g. piezoelectric sensor 11) at step 2206 and checking if the pre-load by the pressure sensor 3 is met at 2208. Referring to FIG. 11B again, the pre-load 1114 is shown in ranges labeled as L for low, M for medium, and H for high. If the pre-load is not met, the system checks if the head is released at step 2210. If the head is released, the system goes back to reading the pressure sensor at step 2202. If the head is not released, the system goes back to acquire reading of the piezoelectric sensor 11 at 2206 and checking if the pre-load is met at 2208. This acquiring reading with a piezoelectric sensor is a pre-test. The head is applied to a treatment point with the pre-load, which is a first compressive force for the pre-test. If the head is released, the probe tip is accelerated into the patient's tissue for the pre-test application of a compressive force. After the probe tip contacts the patient's tissue with a compressive force, the resulting waveform sensed by the treatment head is used to calculate a treatment frequency at step 2212.

The method 2200 continues with setting treatment frequency at step 2214 based on the calculated treatment frequency of step 2212. The treatment frequency is determined based upon the sensed resonant frequency from the piezoelectric sensor. The method 2200 then includes running the treatment at step 2216. This entails applying the probe tip of the treatment head against the patient's tissue for the application of percussive therapy according to the set treatment frequency. In certain instances, when running the treatment, the head is applied to the treatment point with a second compressive force that triggers the system to begin the treatment. The method further includes checking if the treatment is complete or the head is released at step 2218. Following the treatment, a post-test compressive force may be applied. The system may receive the resulting wave form, similarly to the pre-test. The pre-test waveform may be compared to the post-test waveform in order to determine if additional treatment is needed or desired, as will be described subsequently.

The treatment protocols for joint treatment and analysis may include specific treatment plans for each joint. The specific treatment plan may include a number of mapped anatomical locations or spots for treatment; for example, the treatment plan may include five locations for treatment at each joint area. For each of the locations within the treatment plan, the probe tip may be applied to a target spot, and the probe tip may remain in the same position until the post-test is completed. The target spot may also be referred to a target site, or a treatment point. Keeping the probe tip in the same spot on the patient's body for the pre-treatment test, treatment, and post-treatment test can increase the reliability of the treatment dramatically. For instance, the reliability may be increased to about 90% or higher. For any joint in the body, the same application process can be used.

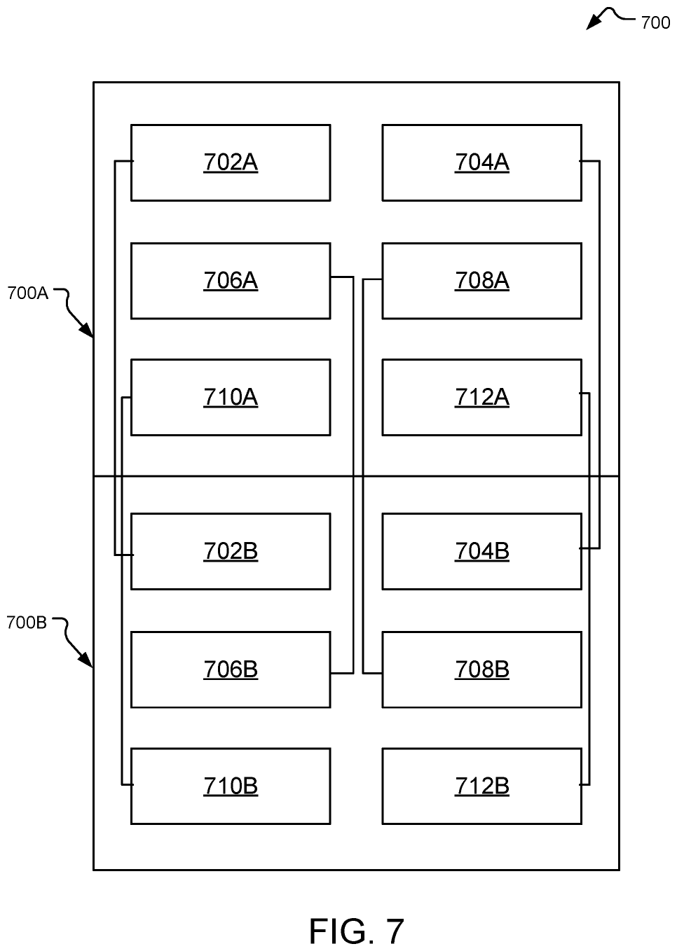

FIG. 7 is a schematic diagram illustrating a module for joint analysis in accordance with an embodiment of the disclosure. As shown in FIG. 7, the joint module 700 includes a joint treatment module 700A including six sub-modules 702A, 704A, 706A, 708A, 710A, and 712A for measurement, treatment, and analysis of shoulder, elbow, wrist, hip, knee, and ankle, respectively. The joint module 700 includes a patient-education module 700B including six sub-modules 702B, 704B, 706B, 708B, 710B, and 712B for patient education of shoulder, elbow, wrist, hip, knee, and ankle, respectively, which may be linked with the respective sub-modules 702A, 704A, 706A, 708A, 710A, and 712A for measurement and analysis of shoulder, elbow, wrist, hip, knee, and ankle so that the education modules are displayed concurrently with treatment of the corresponding joints.

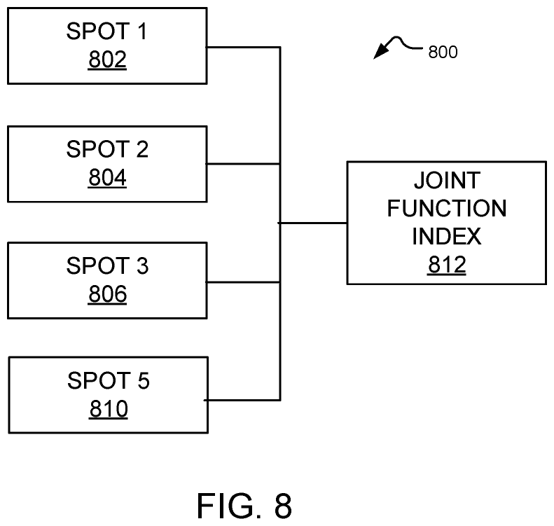

FIG. 8 is a schematic diagram illustrating improvement analysis module 800 after treatment for a joint in accordance with an embodiment of the disclosure. As shown in FIG. 8, for each of the sub-modules of FIG. 7, each of the sub-modules performs pre-test analysis, post-test analysis for each of five spots 802, 804, 806, 808, and 810. A join function index 812 can be obtained based upon the analysis of the five spots.

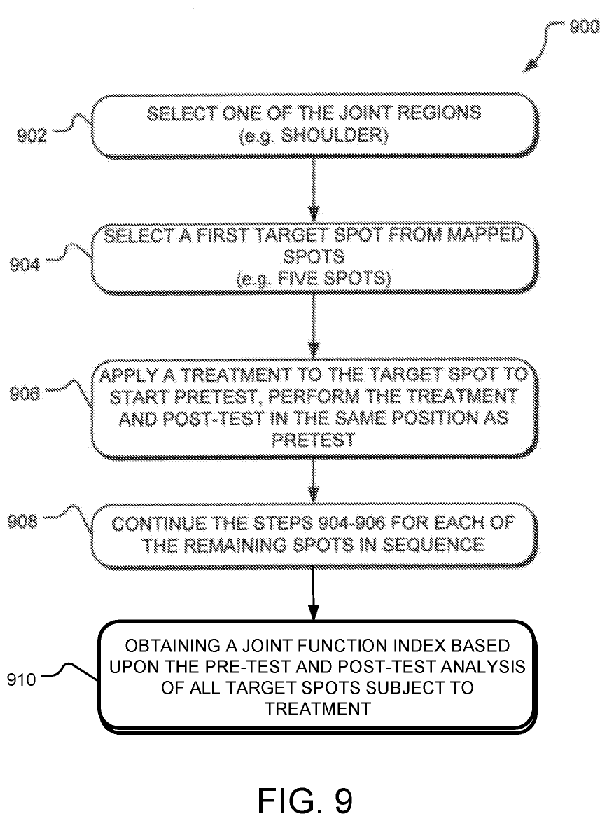

FIG. 9 is a flow chart illustrating the steps of performing the treatment for the joint in accordance with an embodiment of the disclosure. As shown in FIG. 9, a method 900 for treatment of the joint may include selecting one of the joint regions, such as shoulder, elbow, wrist, hip, knee, and ankle, at operation 902, and then may select a first target spot from a number of mapped spots at operation 904. In some variations, each joint has five mapped target locations or sites. The selection of the mapped target locations relates to the joint and the joint mobility. For example, five mapped target locations for a particular joint may be dispersed from each other so as to at least partially encircle the joint of interest. In one example, there may be five points on a shoulder as follows: anterior point; posterior point; superior point; a lateral point; and an inferior point. In this way, joint mobility of the shoulder is tested and treated in an anterior-posterior direction via compressive forces against the anterior and posterior points, in a superior-inferior direction via compressive forces against the superior point and the inferior point, and in a medial direction via compressive forces against the lateral point (for which the glenoid opposes in a lateral direction). For testing and treating of each of the joints, the measurements (e.g., curves, graphs) may be related to the joint spaces and the physical movement of the bones of the joint, like a motion X-ray. The motion X-ray is a correlative test. In this way, the treatment protocols described herein are designed to increase the movement/mobility of the joint, and are achieved at least in part by the treatment spots being located around a joint. This is in contrast to treatment plans which are designed to only stimulate soft tissue, and treat a localized area that does not surround a joint. The protocols described herein are designed to move the boney anatomy along as many axes as possible to stimulate movement of the joint.

The number of target locations for treatment may vary with the region of the joint. Based upon the clinical experiences, five or more locations were found to be adequate for testing and treating the joint mobility of the shoulder along the anterior-posterior axis, the superior-inferior axis, and the medial-lateral axis. While the system may use few or more locations for testing and treatment, five locations are used for all the joints (shoulder, elbow, wrist, hip, knee, and foot ankle) in the treatment protocols.

Referring to FIG. 9, the method 900 may also include applying the treatment head to the target location or spot to start the pre-treatment test (otherwise known as a pre-test) at operation 906. This step may include determining a point-specific treatment plan based on the results of the pre-treatment test, and treating the patient's joint at the particular target location via the percussive force impulse according to the point-specific treatment plan. Finally, this step may also include applying the treatment head to the target location to start the post-treatment test (otherwise known as a post-test). It is noted that the pre-test, treatment, and post-test all occur with the probe tip of the device in the same target location. And, each of the pre-test, treatment, and post-test may commence with depressions of the probe tip that is sensed by the pressure sensor (i.e., pre-test begins with the first depression of the probe tip, treatment begins with the second depression of the probe tip, and post-test begins with the third depression of the probe tip).

For example, a user can apply a treatment head to a first spot of the joint for pre-test, treatment, and post-test. The treatment head may be applied to the first spot. That is, the probe tip is depressed a first time such that it delivers a force impulse to the first point according to the pre-test parameters. Based on the resultant waveform sensed by the device in the pre-test, the instrument can automatically determine the resonant frequency and thus determine a treatment frequency for adjustment. The treatment may begin by depressing the probe tip against the first spot to signal the start of the treatment. After the adjustment shuts off, the user may lift up the probe tip a bit from the adjustment position. The post-test may begin by depressing the probe tip a third time against the patient at the first spot; this will cause the device to deliver a force impulse to the first point, similar to the pre-test, and to receive back a resulting waveform associated with the treated area. The treatment head still remains in the same position on the first spot during all stages.

After the post-test, the measurement and analysis screen reveals a pre-wave analysis and a post-wave analysis of the target spot. Then, the measurement and analysis screen can automatically select the second spot to treat by highlighting the second spot with a circle. The treatment head can then be placed in the second spot as illustrated. When the treatment head is applied to the second spot, the pre-test starts. The system can automatically calculate the resonant frequency of the joint for the adjustment or treatment, and starts the adjustment in the second spot, then shuts off on its own. After the adjustment ends, the user may lift up the treatment head a bit, and when the head is applied again, the post-test begins. The treatment head still remains in the same position on the second spot during the post-test. The position of the treatment head does not change during the pre-test, treatment, and post-test.

The method 900 may further include continuing the steps of 904 and 906 for each of the remaining target locations or spots at operation 908. The user may continue the same routine with the remaining the third, fourth and fifth spots.

The method may also include obtaining a joint function compliance or joint function index based upon the pre-test analysis and post-test analysis of all the target spots at operation 910. After the five spots are all adjusted with the treatment head, the system can calculate the joint function index, which is a measure of overall improvement in joint mobility of the treated joint. More particularly, the system may calculate the differences between the pre-and post-waveforms associated with each treated point. The joint function index may be a summation of the differences between the pre- and post-waveforms associated with each treated point. The joint function index may consider the amplitude changes of the waveform, the area under the curve(s), and the phase-shift, among other factors.

The joint function index may serve as a holistic reference for the mobility of the entire joint. This is in contrast to only testing and treating only a single location on a joint, for instance. Since points are tested and treated in locations that surround the joint (along the plurality of axes described herein), the system is able to obtain a comprehensive determination of joint mobility as indicated by the joint function index.

A low relative reading of the joint function index may indicate that another round of treatment is needed at the particular joint. In contrast, a high relative reading may indicate that the joint experienced a high increase in joint mobility.

### IV. Graphical User Interface for Selecting Treatment Protocols and Delivering Treatments for Increasing Joint Mobility

The system 1111 shown and described with reference to FIGS. 1 and 2 may be used to increase joint mobility of the vertebrae of the spine and the extremity joints (e.g., shoulder, elbow, wrist, ankle, knee, and hip). The treatment protocols may be uniquely tailored for the spine and the individual extremity joints. And, treatments of the spine and the extremity joints may be separate as a practical application of the system 1111 in a clinical setting. For at least these reasons, the description of the system 1111 and the treatment head 44 as applied to the spine is separated from the extremity joints. Section A. describes the system 1111 and treatment head 44 as applied to the extremity joints, and Section B. describes the system 1111 and treatment head 44 as applied to the spine.

### A. Joint Protocol—Shoulder, Elbow, Wrist. Ankle, Knee, and Hip

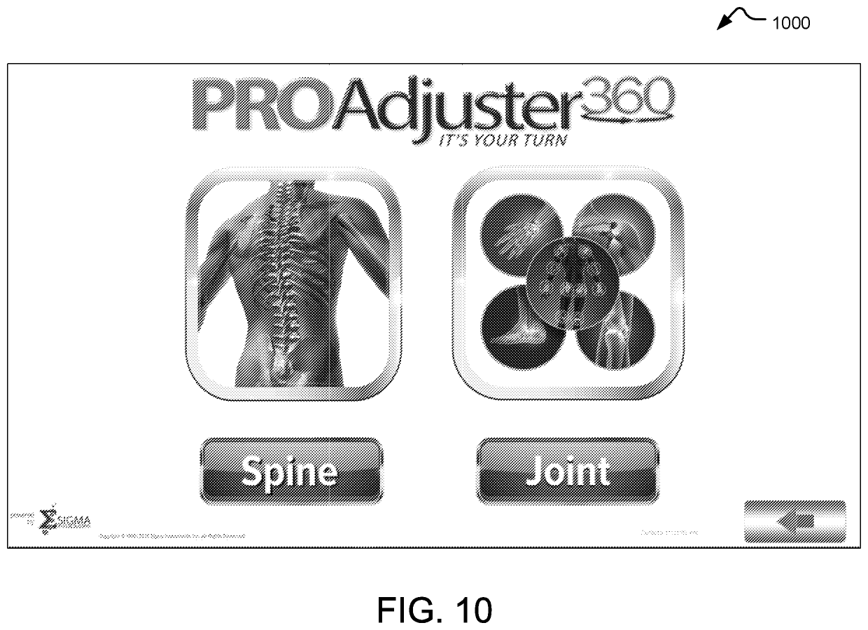

FIG. 10 is an embodiment of a graphical user interface (“GUI”) for the system. In particular, a home screen for the system is displayed. On a home screen as shown in FIG. 10, a screen 1000 includes a touch screen button for joint and a touch screen button for spine. The screen 1000 also shows a sketch of spine above the spine button and a sketch of various joint regions above the joint button. The touch screen buttons may also be referred to as boxes or icons.

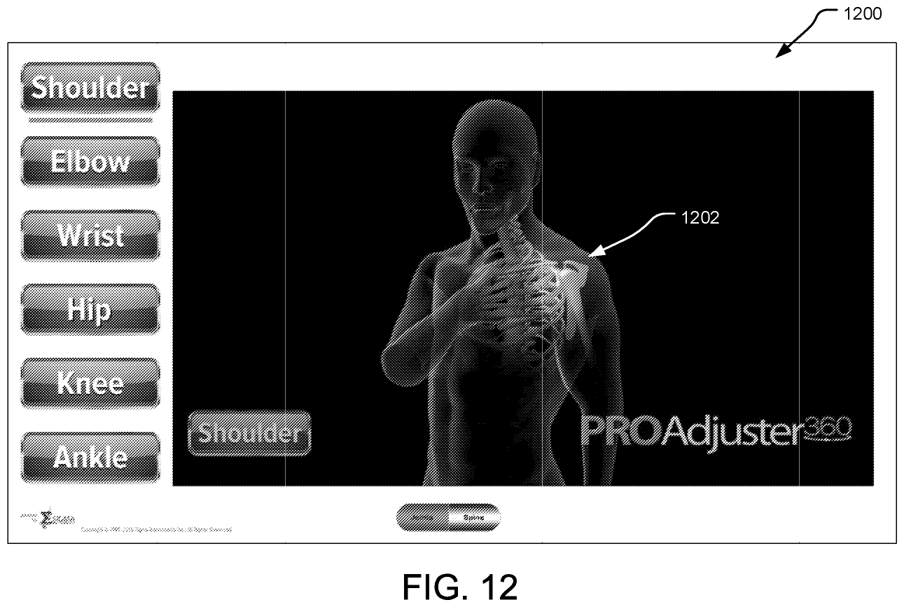

When the joint button is selected (e.g., touched) on the home screen 1000, a patient education screen 1100A is shown in FIG. 11A. FIG. 11A is an embodiment of a patient-education screen displaying particular joint regions for selection. As shown on the left side of the screen 1100A, there are touch screen buttons for the shoulder, elbow, wrist, hip, knee, and ankle. As an example, when the touch screen button for shoulder is selected, the next patient-education screen 1200 is shown in FIG. 12. FIG. 12 is an embodiment of the patient-education screen displaying that shoulder is selected. The patient-education screen 1200 illustrates a highlighted shoulder 1202 for treatment.

The next measurement and analysis screen 1100B following the home screen of FIG. 10 is shown in FIG. 11B. FIG. 11B is an embodiment of a measurement and analysis screen on another side. The measurement and analysis screen 1100B shows a body sketch 1102 including various joints highlighted in circular areas 1104 on the left side. The measurement and analysis screen 1100B also includes a limit indicator 1106 for the number of force impacts, a force indicator 1108, and a frequency indicator on the right side. For example, as shown in FIG. 11B, the force applied by the treatment head is 10 lbs. at a frequency is 8 Hz with a limit of 50. The limit of 50 is equivalent to 6.25 seconds. The treatment head will shuts off after the limit of 50 or 6.25 seconds. As another example, when a frequency of 10 Hz is used, the limit of 50 is equivalent to 5 seconds. The treatment head will shuts off after the limit of 50 or 5 seconds.

The system may include two screens arranged side by side, a first patient education screen as shown in FIG. 11A, and a second measurement and analysis screen showing the analysis of the selected joint, as shown in FIG. 11B. Alternatively, the patient education screen in FIG. 11A and the second measurement and analysis screen of FIG. 11B may be shown on a single screen. Alternatively, only one of the patient education screen in FIG. 11A and the second measurement and analysis screen of FIG. 11B may be shown at a time. Alternatively, the patient education screen of FIG. 11A may be omitted from the system 1111.

Referring to FIG. 11B, the system can select different ranges of the sub-harmonic frequency based on the pre-test, or the practitioner can select/adjust one of the treatment parameters. For example, the system allows the selection of different ranges within a harmonic frequency 1110 (e.g. 12 Hz) for a particular joint region. This sub-harmonic frequency may relate to bone density. For example, a patient of age sixty-two may have a lower bone density than a young adult of age twenty-two.

In some variations, the system may provide three different ranges of sub-harmonic frequencies. The system may include a mode for selecting one of the three different ranges of sub-harmonic frequencies. Referring still to FIG. 11B, the mode 1112 includes α, θ, δ for three different sub-harmonic frequencies.

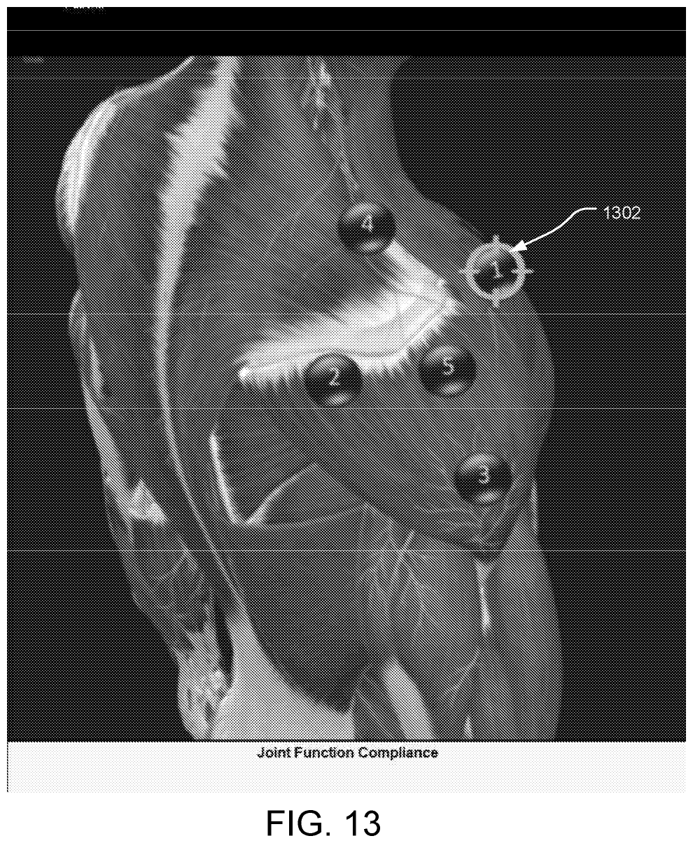

From the screen in FIG. 11B, the practitioner may select a joint region 1104 from the body 1102, which opens up the screen of FIG. 19 depicting a specific joint treatment protocol for the shoulder. Similarly, selecting a different joint region from the body 1102 will open up a screen with a specific joint treatment protocol for the selected joint. FIGS. 13-18 show the five spots labeled as 1-5 for shoulder, elbow, wrist, knee, hip, and ankle, respectively, which are shown on the measurement and analysis screen. It is appreciated that while the depictions of the musculature in FIGS. 13-18 are shown without the full display screen from FIG. 19, the full display screen and its functionality is present. That is, FIGS. 13-18 only show the left portion of the display screen of FIG. 19. FIGS. 13-18 only show the left portion of the display screen with the musculature and treatment points in order to clearly show the location of the treatment points.

For the shoulder, as seen in FIGS. 13, and 19-20, the locations of the points are as follows: point 1 is located on or approximately the anterior deltoid; point 2 is located on or approximately the posterior deltoid just inferior of the attachment of the deltoid to the acromion of the scapula; point 3 is located on or approximately the lateral deltoid at the deltoid tuberosity or just superior to the deltoid tuberosity; point 4 is located on or approximately the lateral aspect of the middle trapezius just superior of the attachment of the trapezius to the clavicle; and point 5 is located on or approximately the lateral deltoid just inferior of the attachment of the deltoid to the acromion of the scapula. These particular points are intended to provide movement to the joint, upon treatment, along various axes to accurately determine the extent to which treatment effects the mobility of the joint.

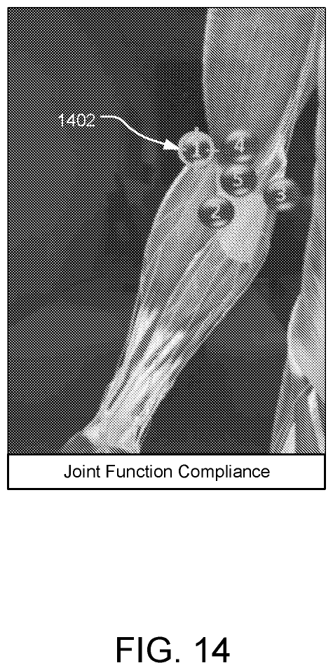

For the elbow, as seen in FIG. 14, the locations of the points are as follows: point 1 is located on or approximately the brachioradialis and/or the brachialis just lateral of the biceps brachii; point 2 is located on or approximately just distal the biceps brachii tendon near an overlap of the pronator teres and the brachioradialis; point 3 is located on or approximately at the interconnection of the common flexor tendon and the medial epicondyle of the humerus (the common flexor tendon serves as a proximal attachment point for the superficial muscles of the front forearm the flexor carpi radialis, palmaris longus, pronator teres, flexor digitorum superficialis, and flexor carpi ulnaris; point 4 is located on or approximately the biceps brachii just proximal of the biceps brachii tendon; and point 5 is located on or approximately the biceps brachii tendon. To provide treatment along multiple axes, the treatment head may be angled medially for point 1, and laterally for point 3, as an example.

For the wrist, as seen in FIG. 15, the locations of the points are as follows: point 1 is located on or approximately at an interconnection point between the radius and the central carpal bones; point 2 is located on or approximately an interconnection point between the ulna and the lunate; point 3 is located on or approximately an interconnection point between the radius and the scaphoid at the wrist joint; point 4 is located on or approximately the distal end of the ulna; and point 5 is located on or approximately the distal end of the radius. Generally, points 1, 2, and 3 extend across the wrist along the wrist joint line between the radius and ulna on an arm side, and the proximal row of carpal bones on the hand side. Alternatively, the points 1, 2, and 3 may extend across the midcarpal joint between the distal row of carpal bones and the proximal row of carpal bones.

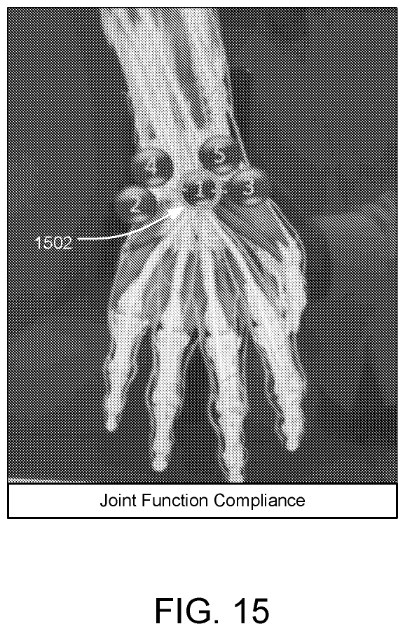

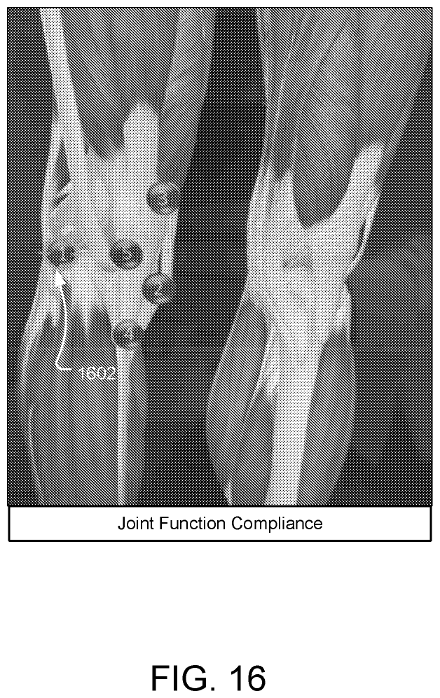

For the knee, as seen in FIG. 16, the locations of the points are as follows: point 1 is located on or approximately the lateral hamstring tendon; point 2 is located on or approximately the Sartorius on a medial side of the knee; point 3 is located on or approximately the semimembranosus; point 4 is located on or approximately the plantaris or a proximal portion of the gastrocnemius on a posterior side of the knee joint; and point 5 is located on or approximately the posterior side of the knee near the opening formed by the coming together of the many muscles of on the posterior side of the knee including the medial and lateral heads of gastrocnemius muscle, the biceps femoris, the plantaris, and the semimembranosus muscles.

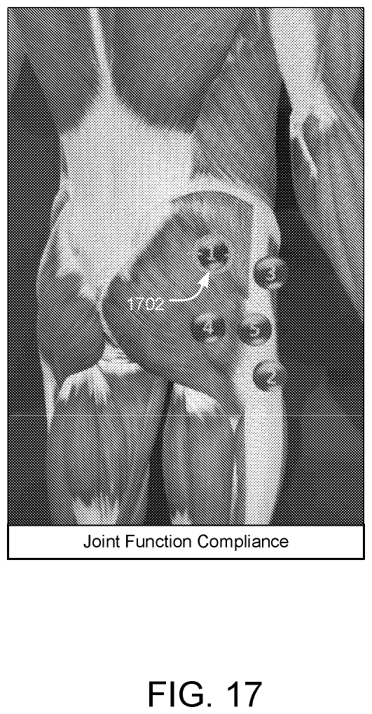

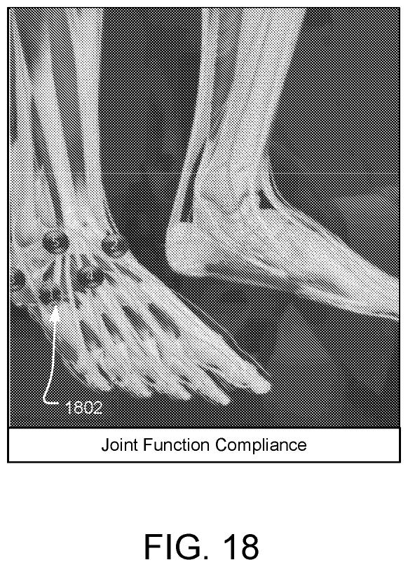

For the hip, as seen in FIG. 17, the locations of the points are as follows: point 1 is located on or approximately an intersection point of the gluteus maximus and the gluteus medius; point 2 is located on or approximately the iliotibial band or tract; point 3 is located on or approximately an interconnection of the iliotibial band or tract and the tensor fasciae latae, opposite the gluteus medius; point 4 is located on or approximately the gluteus maximus; and point 5 is located on or approximately the iliotibial band or tract just superior to point 2. For the ankle, as seen in FIG. 18, the locations of the points are as follows: point 1 is located on or approximately the dorsal calcaneocuboid ligament; point 2 is located on or approximately the medial ankle joint in the vicinity of the anterior tibiotalar part of the medial ligament and the tibionavicular part of the medial ligament; point 3 is located on or approximately the calcaneofibular ligament; point 4 is located on or approximately dorsal talonavicular ligament; and point 5 is located on or approximately the anterior talofibular ligament.

As shown in FIG. 13, the screen reveals a selection of one spot at a time, for example, the selected spot for the shoulder is highlighted with a circle 1302 in FIG. 13. The particular highlighted spot is on an anterior portion of the shoulder.

When a user wants to treat the elbow, for example, the user may touch the elbow icon from the body 1102 on FIG. 11B to bring up the elbow screen as shown in FIG. 14, which depicts five specific locations for treatment to increase joint mobility. Spot 1 is highlighted with a circle 1402. When a user wants to do treatment for the wrist, the user may touch the wrist icon on FIG. 11B to bring up the wrist screen as shown in FIG. 15. Spot 1 is highlighted with a circle 1502. When a user wants to do treatment for the knee, the user may touch the knee icon on FIG. 11B to bring up the knee screen as shown in FIG. 16. Spot 1 is highlighted with a circle 1602. When a user wants to do treatment for the hip, the user may touch the hip icon on FIG. 11B to bring up the hip screen as shown in FIG. 17. Spot 1 is highlighted with a circle 1702. When a user wants to do treatment for the ankle, the user may touch the ankle icon on FIG. 11B to bring up the ankle screen as shown in FIG. 18. Spot 1 is highlighted with a circle 1802. For each of the joints subject to treatment, the practitioner may perform the treatment for the five spots in sequence.

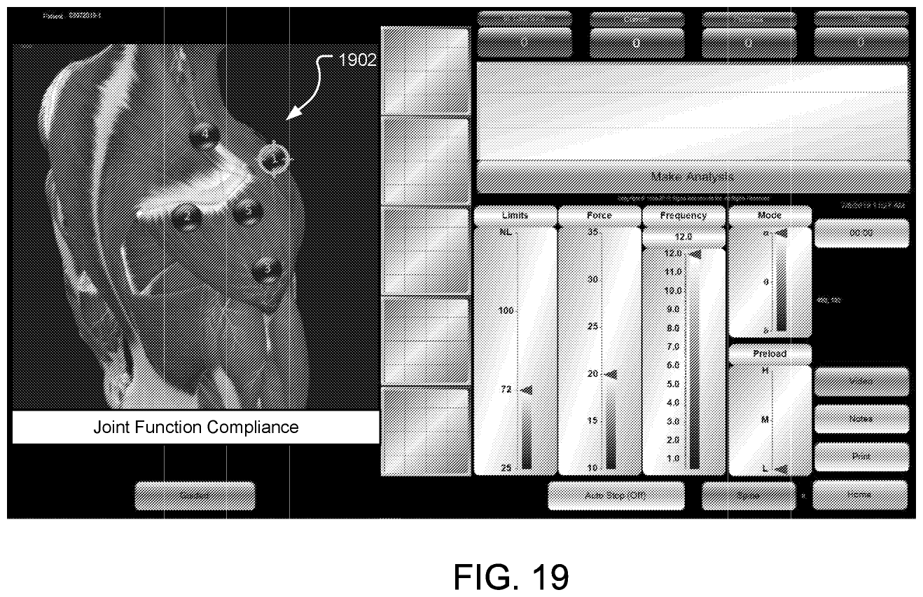

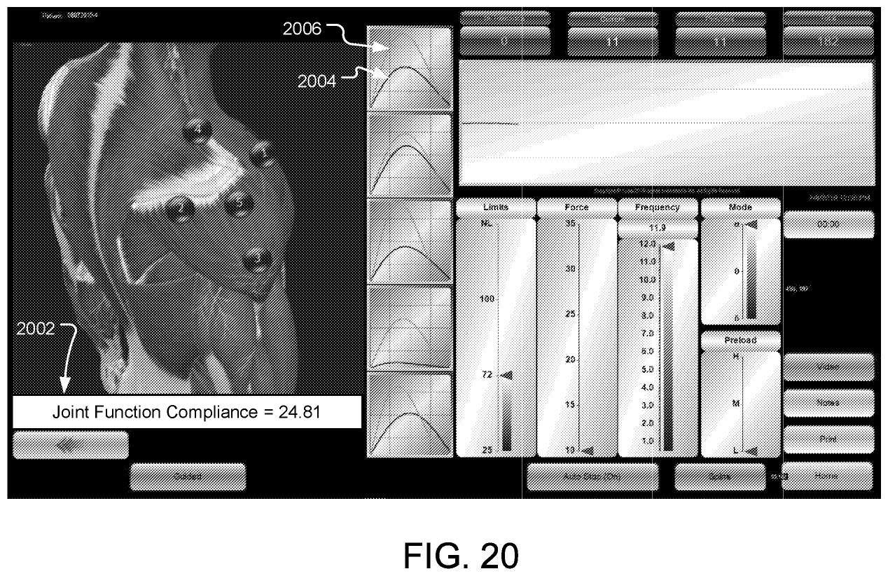

An example treatment protocol for the shoulder is illustrated in FIGS. 19 and 20. The protocol may be duplicated for the other points on the joints shown in FIGS. 14-18. FIG. 19 is an embodiment of the measurement and analysis screen displaying the first spot selected for shoulder and the force indicator, frequency indicator, the limits of impacts, mode indicator, and preload indicator. As shown in FIG. 19, when the shoulder is selected, the measurement and analysis screen shows the shoulder with one of the five spots selected with a circle. As shown on the screen, spot 1 is selected. The particular parameters may be shown for the pre-test as this is the first use of the treatment device on the shoulder. As seen in FIG. 19, the limit for the number of impacts is 72, the force is 20 lbs., and the frequency is 12 Hz. These parameters may be adjusted by the practitioner.

All five analysis spots, in FIG. 19, can be treated according the method 900 shown in FIG. 9. In sum, the treatment head 44 may be applied to the first location on the patient's body by depressing the probe tip against the first location. This triggers the pre-treatment test according to the parameters shown on FIG. 19. The resulting waveform sensed by the treatment head 44 is received by the system 1111, and the system 1111 determines treatment parameters including force, frequency, limits of impact, and mode based on the resulting waveform (e.g., a subharmonic frequency of the waveform). Next, the treatment head 44 may be depressed a second time against the first location on the patient to start the treatment. Once the mechanical force impact treatment has finished, the practitioner may release the treatment head 44 from the patient, and again depress the probe tip against the first location to trigger the post-treatment test. The resulting waveform is shown in the form of a wave in FIG. 20. After treatment of the first point, the system 1111 queues up the second point for treatment.

FIG. 20 is an embodiment of the measurement and analysis screen displaying treatment conditions including a force indicator, frequency indicator, and limits of impacts, mode indicator, and preload indicator and the joint function compliance for the shoulder. As seen in FIG. 20, there are five graphs oriented vertically in a line, each one including a pre-test curve 2004 and a post-test curve 2006 resulting from the pre-test and the post-test, respectively. These curves may be similar to the curves shown in FIGS. 3-5. The treatment of all five points has been completed in FIG. 20, and therefore the joint function index 2002 has been computed and displayed. The joint function index or compliance 2002 is calculated based upon the analysis of pre- and post-test curves. In this example, the joint function compliance has a positive value of 24.81.

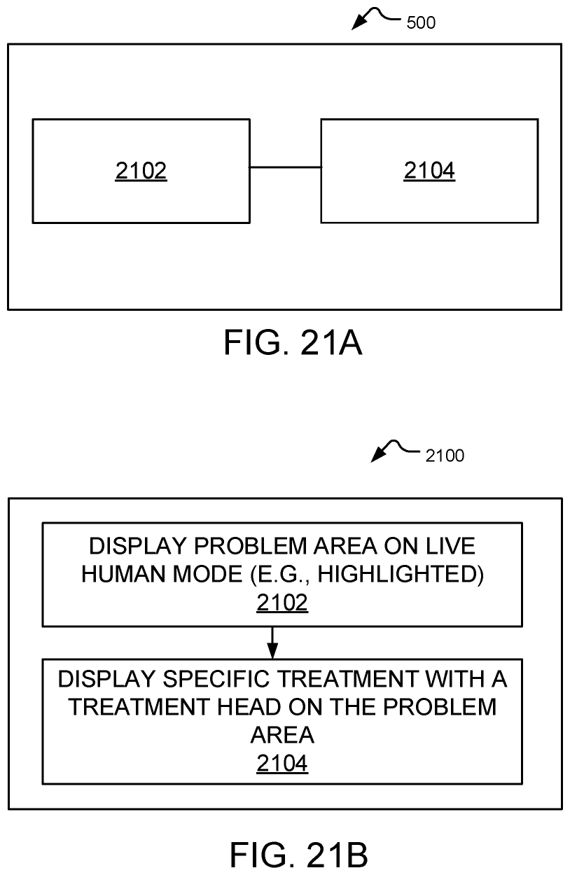

FIG. 21A is an embodiment of a display including two display screens for the joint analysis. As shown in FIG. 21A, a display or a touch screen 500 of FIG. 1 may include a measurement and analysis screen 2102 and a patient education screen 2104. The patient-education screen 2104 may be linked with the measurement and analysis screen 2102. The measurement and analysis screen may be linked with the joint treatment module 700A. The patient-education screen 2104 is tied to the patient-education module 700B for the purpose of explaining to the patient either before or after the treatment. The patient-education module provides visual representation of what is happening during treatment.

FIG. 21B is a flow chart illustrating the educational module for the joint treatment in an embodiment of the disclosure. A method 2100 may include displaying problem area on a live human model on the patient-education screen at operation 2102. For example, the problem area may be highlighted. The method 2100 may also include displaying treatment of the problem area with the treatment head applied on the problem area at operation 2104.

### B. Spine Protocol

The goal of the adjustment of spine is to increase motion or fluid mechanical motion of the spine itself. The adjustment to the spine includes releasing and lining up the spine correctly.

The pretest, treatment, and post-test for the spine can be done by using the treatment head as shown in FIG. 1 that includes a piezoelectric sensor to read the resonant frequency. The treatment head may use a spine tip such as shown in FIG. 6A for the spine treatment and analysis. The system can perform pre-test, pre-test analysis, treatment, post-test, and post-test analysis for the spine.

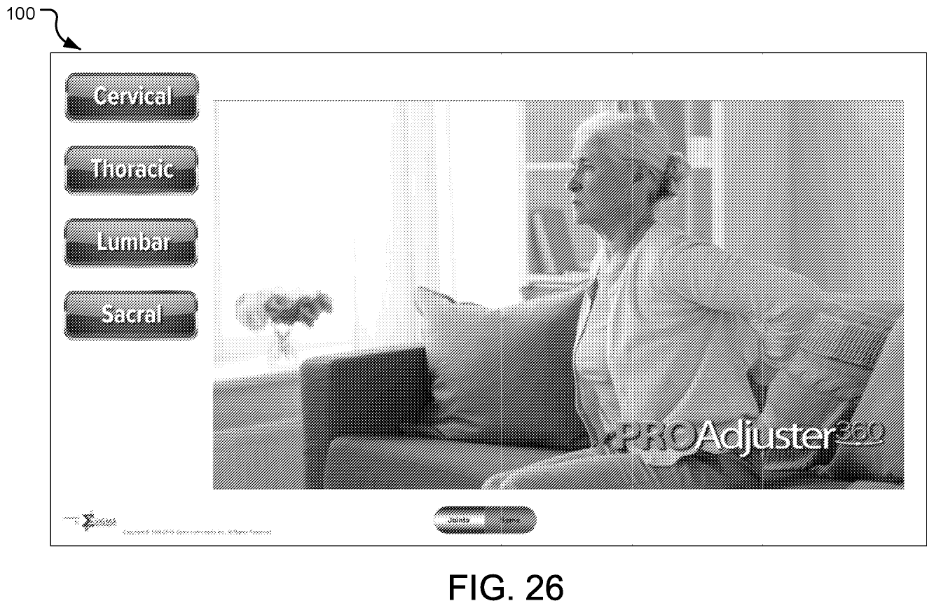

From the screen 1000 in FIG. 10, the practitioner may select the spine button in order to queue up the menu of spinal treatment protocols, which are shown in FIG. 26. As shown in FIG. 26, there are four icons, including cervical, thoracic, lumbar, and sacral for treatments of the cervical spine segment, thoracic spine segment lumbar spine segment, and sacrum spine segment, respectively.

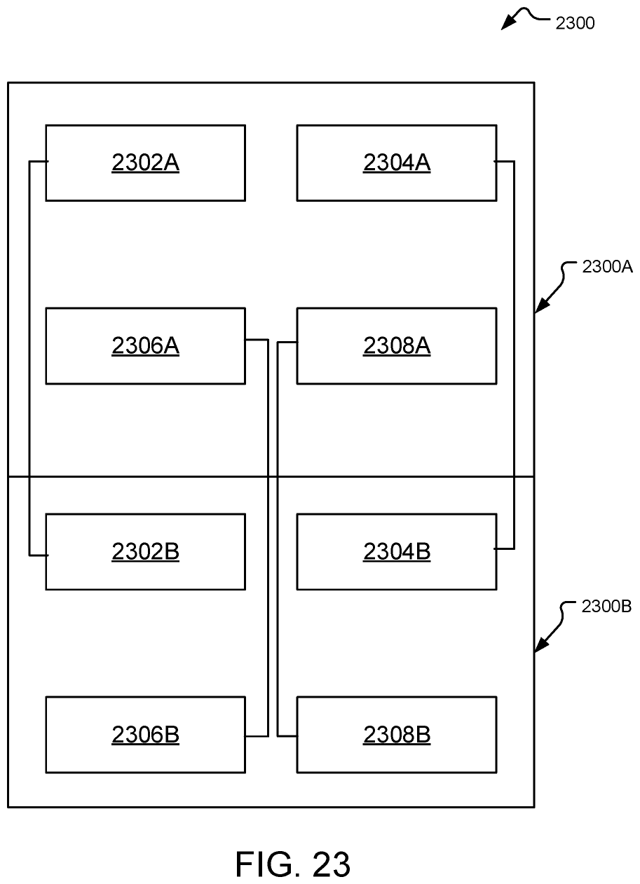

FIG. 23 is a schematic diagram illustrating a spine module for spine treatment and analysis in accordance with an embodiment of the disclosure. As shown in FIG. 23, the spine module 2300 includes a measurement and analysis module 2300A including four sub-modules 2302A, 2304A, 2306A, 2308A for measurement and analysis of cervical spine segment, thoracic spine segment, lumbar spine segment, and sacral spine segment, respectively. The spine module 2300 includes a patient-education module 2300B including four sub-modules 2302B, 2304B, 2306B, 2308B for patient education of cervical spine segment, thoracic spine segment, lumbar spine segment, and sacral spine segment, respectively, which may be linked with the respective sub-modules 2302A, 2304A, 2306A, 2308A for measurement and analysis of cervical spine segment, thoracic spine segment, lumbar spine segment, and sacral spine segment such that the education modules are displayed during treatment of the corresponding portion of the spine.

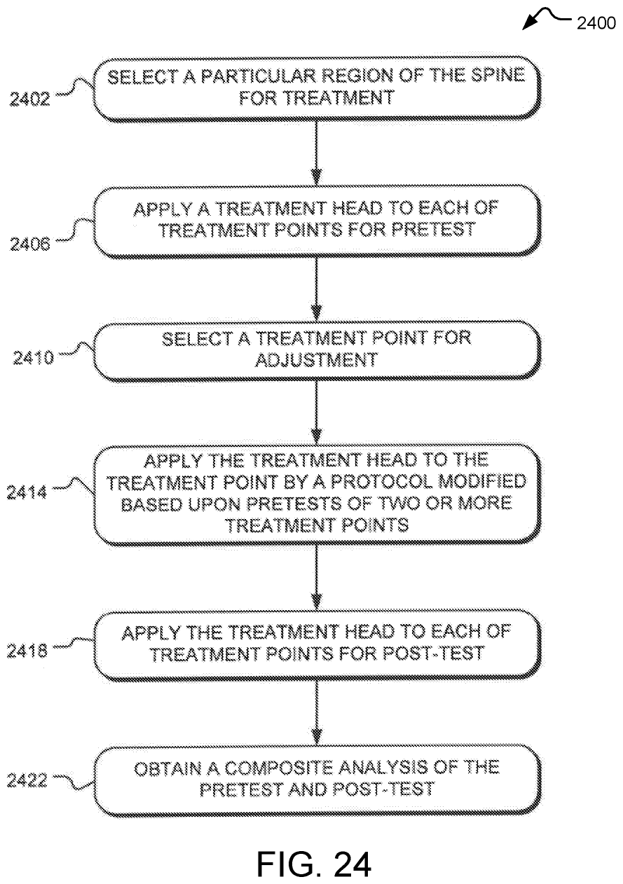

FIG. 24 is a flow chart illustrating the educational module for the spine treatment in accordance with an embodiment of the disclosure. A method 2400 for treating the spine may include selecting a particular region of the spine for treatment at operation 2402 (selection of the region of the spine may be done via the GUI of FIG. 26). The method 2400 may also include applying a treatment head to each of treatment points to pre-test each of the treatment points (each vertebra in the selected region) at operation 2406. The method 2400 may continue with selecting one of the treatment points within the selected region for adjustment at operation 2410. The method 240 may also include applying the treatment head to the selected treatment point with a modified protocol based upon the pre-test of one treatment point, or a modified protocol based upon pre-test of two or more treatment points at operation 2414. In some variations, the modified protocol is based upon the resonant frequency detected at the treatment point. In some variations, the modified protocol is based upon pre-test of two or more treatment points, in which at least one of the treatment points is where the treatment is applied (e.g. a treatment point C2 in cervical spine), and at least one of the treatment points is in another location of the spine, for example, in another spine segment (e.g. a treatment point T5 in thoracic spine segment).

The method 2400 may also include applying the treatment head to all the treatment points at operation 2418. The method 2400 may further include obtaining a composite analysis of the pre-tests and post-tests of all the treatment points. In some variations, one treatment point (e.g. C3) in the cervical spine segment may be affected by the treatment in one treatment point (e.g. T6) of another spine segment, such as thoracic spine segment, among others.

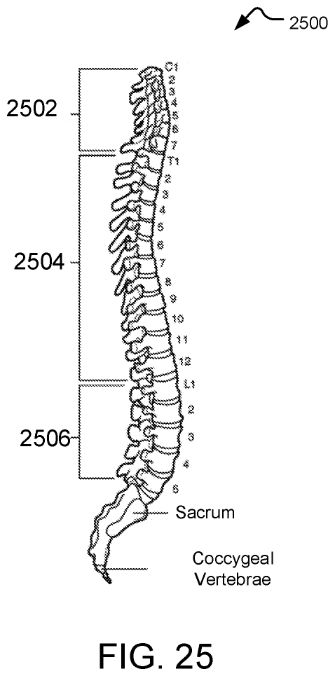

FIG. 25 is a diagram illustrating a full spine displaying treatment spots in accordance with an embodiment of the disclosure. As shown, a full spine 2500 may include cervical spine segment 2502, thoracic spine segment 2504, lumbar spine segment 2506, and sacrum spine segment 2508. The cervical spine segment includes seven cervical vertebrae C1-C7. The thoracic spine segment 2504 includes eleven thoracic vertebrae T1-T11. The lumbar spine segment 2506 includes five lumbar vertebrae L1-L5. The sacrum spine segment 2508 includes five sacrum vertebrae S1-S5. Each vertebra is a potential treatment point.

To begin treatment of the spine, a practitioner may start by touching the spine icon on the home screen of FIG. 10, which brings up the screen of FIG. 26. FIG. 26 is an embodiment of the patient-education screen displaying particular spine regions. When the practitioner touches any one of the four icons, such as thoracic, the thoracic treatment can start. There are 12 treatment points for thoracic spine segment. The user may start with pre-testing on T1, and may continue with pre-testing on T2, T3 . . . till T12. Once the pre-tests at 12 spots are finished, one may select one or more of T1-T12 for adjustment.

The system can calculate the correct hit rate or the adjustment speed which is a sub-harmonic frequency of the resonant frequency that is calculated based upon the pre-test of one treatment point, or based upon the pre-tests of two or more treatment points with the treatment head including the piezoelectric sensor. Each of spots T1-T12 may have a specific sub-harmonic frequency.

The adjustment may be performed at an angle, for example, about 43 degrees from the back of a body. The system can shut off automatically. The user may apply the treatment head to the T1-T12 target spots for a post-test analysis. After the post-tests of all the treatment points are finished, the instrument shows the pre-test results and post test results overlapping for each spot. A composite analysis can be obtained to reveal the improvement of the spine.

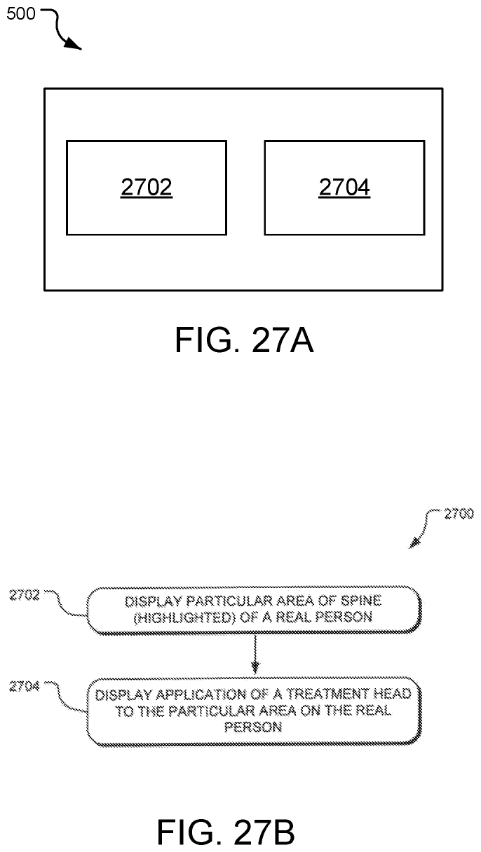

FIG. 27A is an embodiment of a display including two display screens for the spine analysis. As shown in FIG. 27A, a display or a touch screen 500 of FIG. 1 may include a measurement and analysis screen 2702 and a patient education screen 2704. The patient-education screen 2704 is connected or in electrical communication with and the measurement and analysis screen 2702. The measurement and analysis screen is in communication with the measurement and analysis module 2300A, as shown in FIG. 23. The patient-education screen 2704 is tied to the patient-education module 2300B as shown in FIG. 23 for the purpose of explaining to the patient either before or after the treatment. The patient-education module provides visual representation of what is happening.

FIG. 27B is a flow chart illustrating the steps of displaying on the patient-education screen for the spine in accordance with an embodiment of the disclosure. A method 2700 may include displaying problem area on a live human model on the patient-education screen at operation 2702. For example, the problem area may be highlighted. The method 2700 may also include displaying treatment of the problem area with the treatment head applied on the problem area at operation 2704.

### V. Graphical User Interface for Selecting Treatment Protocols and Delivering Treatments of Soft Tissue

The system 1111 of FIGS. 1 and 2 may be used to apply therapy of the soft tissue, such as muscles and ligaments. One purpose of the therapy is to recalibrate the neuro-pathway, so that the soft tissue primarily gets elongated. When muscles get tight, the muscles get tight and shorten. The shortening of the muscle may pull the bones that are attached together. For example, when the muscles are tight in the shoulder, the shoulder may not move as a person may wish, such as when raising the arms. Such tightness may be the results of lack of joint mobility as well as the tightness of the muscles and tissue surrounding the joint. Therefore, the system 1111 of FIGS. 1 and 2 may be to use to lengthen the muscles and soften the muscles, via a very high speed impact with simultaneous movement or manipulation of the muscles. For example, the patient may raise their arm while the treatment head 44 of FIGS. 1 and 2 impacts a target spot of the shoulder at the high speed so as to provide a therapy to the shoulder muscles. The system for treating soft tissue and the system for treating the joints may be used independently from each other or in combination.

The treatment head 44 may include the same elements or be modified from the head 44 shown in FIGS. 1 and 2. For example, the treatment head 44 for soft tissue may be the same as shown in FIGS. 1 and 2, except the device for soft tissue may not include the piezoelectric sensor, among other elements.

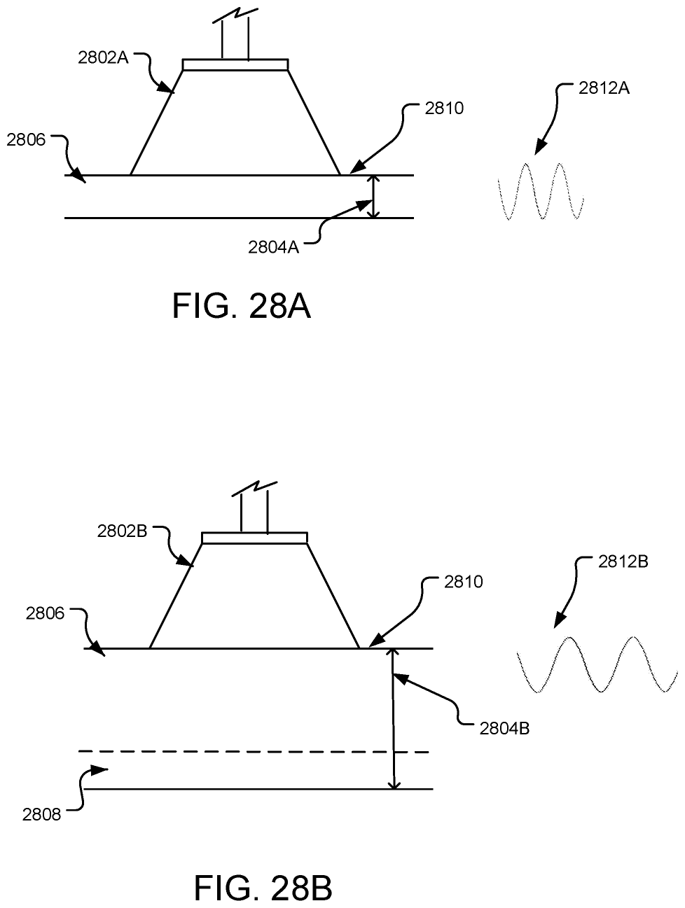

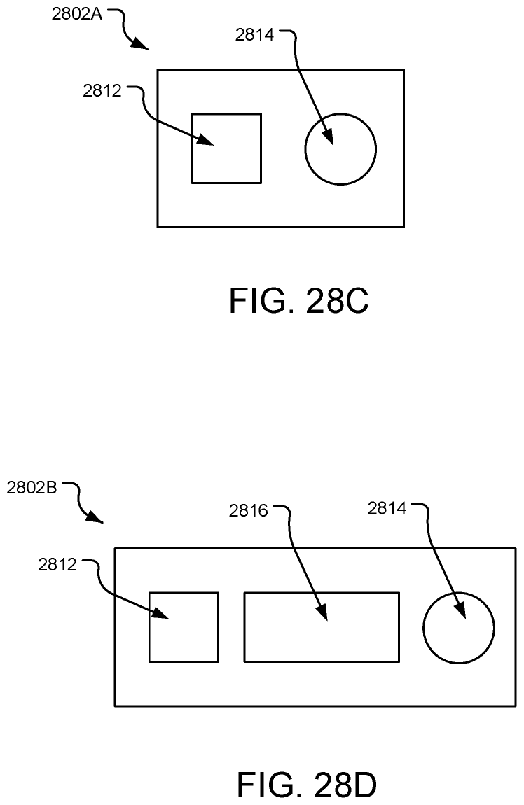

FIG. 28A illustrates the soft tissue treatment head with a higher frequency in accordance with an embodiment of the disclosure. As shown in FIG. 28A, a probe tip 2802A of a treatment head is applied on the soft tissue 2806 having a skin surface 2810. The probe tip 2802A of the treatment head is smaller in size such that the frequency may range from 13 Hz to 30 Hz, which is higher than the treatment head used for the adjustment of joint or spine. This higher frequency does not penetrate as deep as the lower frequency (e.g. up to 12 Hz) for the joint or spine. The depth 2804A is relatively shallow, compared to the penetration depth 2804B shown in FIG. 28B. Thus, the probe tip with smaller size and higher frequency 2812A can be used for therapy of the soft tissue, which is less deep as compared to the bones of the joint or spine.

FIG. 28B illustrates a probe tip of the joint and spine treatment head with a lower frequency in accordance with an embodiment of the disclosure. As shown in FIG. 28B, the probe tip 2802B of the treatment is applied to the skin surface 2810, the bone 2808 of the joint or spine is underneath the skin surface 2810. The probe tip 2802B of the treatment head is larger than the probe tip of the treatment head 2802A and generates a lower frequency wave 2812B than the wave 2812A. The penetration depth 2804B is deeper than the penetration depth 2804A for the treatment head used for treating soft tissue.

FIG. 28C shows a schematic diagram of a treatment head without a piezoelectric sensor in the treatment of the soft tissue of FIG. 28A in accordance with an embodiment of the disclosure. As shown in FIG. 28C, the treatment head 2802A includes a probe 2812 and a pressure sensor 2814. In contrast, the treatment head 2802B of FIG. 28D includes a probe 2812 and a pressure sensor 2814, and a piezoelectric sensor 2816. It is noted the probe in FIG. 28D is a schematic diagram of a treatment probe such as shown in FIGS. 1 and 2. The treatment head 2802A used for the soft tissue does not include the piezoelectric sensor 2816 as used in the treatment head for the adjustment of joint or spine.

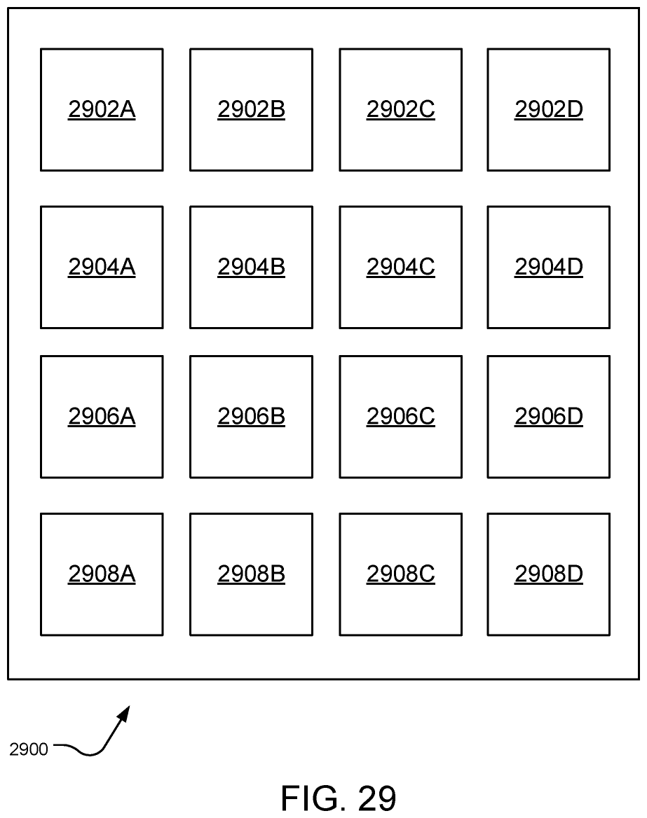

FIG. 29 is a schematic diagram illustrating a soft tissue module for the soft tissue treatment in accordance with an embodiment of the disclosure. As shown in FIG. 29, a soft tissue treatment module 2900 may include four sub-modules 2902A, 2902B, 2902C, and 2902D for shoulder, elbow, wrist and hand, respectively, which are in a first group of upper extremity. The soft tissue treatment module 2900 may include four sub-modules 2904A, 2904B, 2904C, and 2904D for hips, knee, ankle, and feet, respectively, which are in a second group of lower extremity. The soft tissue treatment module 2900 may include four sub-modules 2906A, 2906B, 2906C, and 2906D for neck, lower back, pelvis, ribs, respectively, which are in a third group of spines, ribs, and pelvis. The soft tissue treatment module 2900 may also include four sub-modules 2908A, 2908B, 2908C, and 2908D for balance, performance, golf, and freedom, respectively, which are in a fourth group of general health.

The therapy for the soft tissue is based upon a predetermined protocol for each target location of the tissue. The goal of the therapy is to soften the tissue. Before the treatment, the tissue may be tight. After the treatment, the tissue may be softened.

The therapy is a mechanical motion therapy, which is a dynamic process, and is different from the static adjustment of the joint or spine. The therapy is dynamic because it is designed to be utilized in conjunction with patient' movement/stretching. Thus, treatment of the soft tissue is maximized by the combined effort of percussive massage at specific points on the body, along with active movements of the body in or around the treated area.

The system is an icon-driven therapy system, which is user friendly. Any staff members or doctors can perform the therapy by performing the protocols. The therapy included selected protocols which set the limits of how many times the treatment head would hit or tap, and also sets the force and the frequency. The predetermined protocols are based upon experiences, research, and clinical application of mechanical motion therapy in thousands of applications. The particular parameters of the selected protocols are specifically tailored for the location of treatment, and the motion activity to be performed. Therefore, the particular parameters may be different depending on the soft tissue to be treated and the motion to be performed.

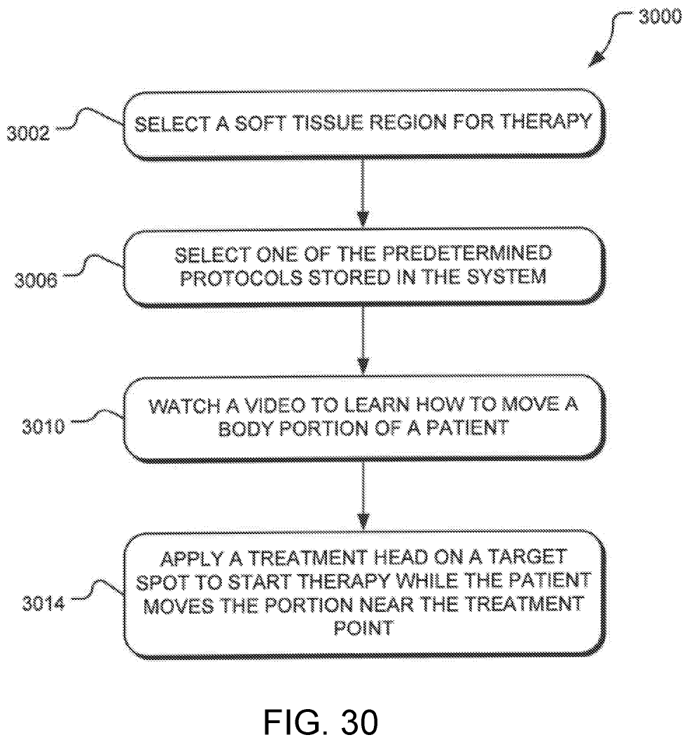

FIG. 30 is a flow chart illustrating the steps of performing the treatment for the soft tissue in accordance with an embodiment of the disclosure. A method 3000 for treating the soft tissue may include selecting a soft tissue region for therapy at operation 3002. The method 3000 may also include selecting one of the protocols stored in the system at operation 3006. The method 3000 may also include watching an instructional video to learn how to move a body portion of a patient at operation 3010 as the therapy treatment may be applied while the patient is performing the movement from the video. The method 3000 may further include placing a treatment head on a target spot to start therapy while the patient moves the body portion near the target spot as instructed in the video at operation 3014. When the screen indicates “continue to treatment,” one may press the treatment head to the target spot to continue.

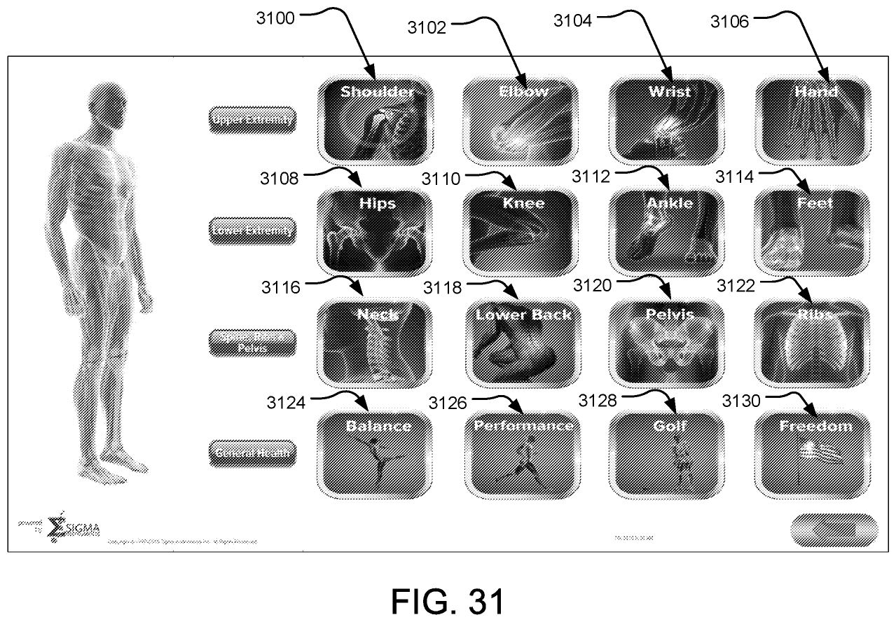

As an example, a practical application of the use of the system is described below. FIG. 31 shows a home screen with four rows including four icons, i.e. (1) upper extremity, (2) lower extremity, (3) spine, ribs, and pelvis, and (4) general health. For each of the rows, there are four icons. There are a total of sixteen icons for different body parts, including shoulder, elbow, wrist, hand, hips, knee, ankle, feet, neck, lower back, pelvis, ribs, balance, performance, golf, and freedom, as shown in the screen shot of FIG. 31.

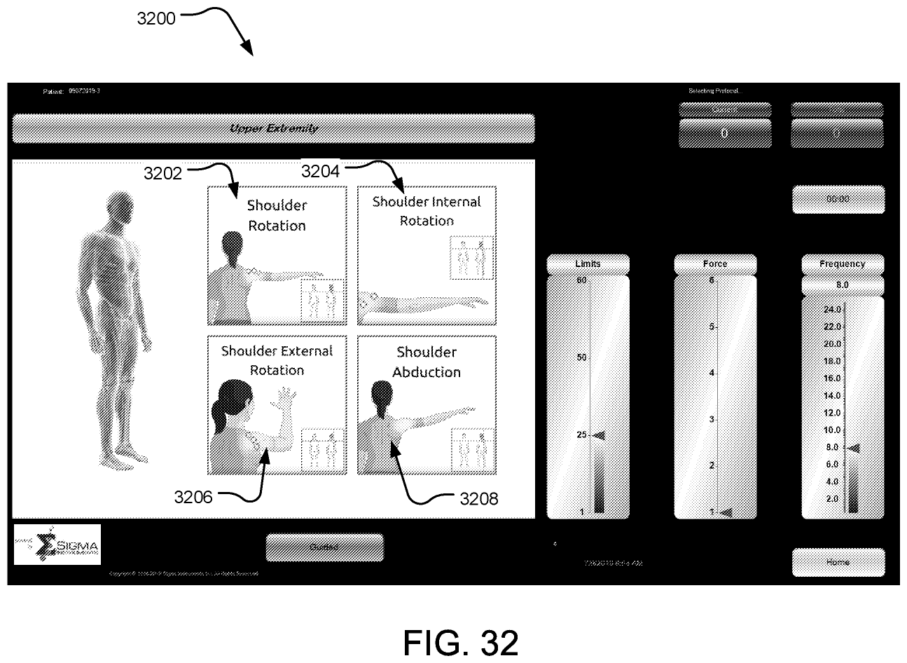

As an example, a shoulder button 3100 is selected from FIG. 31, which opens up the shoulder protocol selection screen 3200 shown in FIG. 32. From this screen 3200, the user may select from four shoulder protocol modules: shoulder rotation 3202; shoulder internal rotation 3204; shoulder external rotation 3206; and shoulder abduction 3208. Upon selection of one of the protocols, another screen will appear with selection options for running that particular protocol. As seen in FIG. 32, the shoulder internal rotation module 3204 includes treatment points on the posterior deltoid and the lateral deltoid. The shoulder external rotation module 3206 includes treatment points on the superior edge of the deltoid where it meets the trapezius. The shoulder abduction modules includes treatment to the general area of the posterior shoulder. In certain instances, the treatment points may be in other locations.

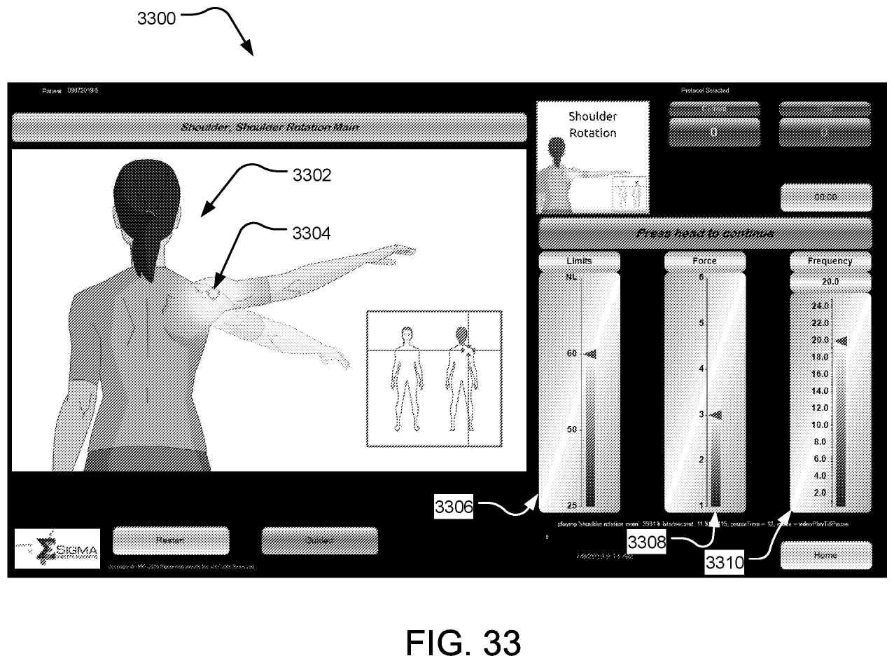

When the shoulder rotation protocol 3202 is selected, from FIG. 32, a shoulder rotation protocol navigation screen 3300 appears, as seen in FIG. 33. The screen 3300 includes an image of a representative patient 3302 in animated motion of the shoulder. More particularly, the representative patient 3302 depicts the shoulder motion to be performed by the patient during this particular treatment. In the case of the shoulder rotation protocol, the animated image of the representative patient 3302 rotates the shoulder inward. Highlighted at point 3304 on the posterior deltoid of the representative patient 3302 is a location of treatment points for applying the treatment head.

As seen in FIG. 33, the navigation screen 3300 for the shoulder rotation module includes pre-set parameters for the treatment, including the limit, the force, and frequency for treatment to a shoulder at this point and while the patient performing the animated motion. The limit is defined as the maximum number of percussions per spot. The Force is defined as how hard the treatment head percusses during treatment. And frequency is defined as how fast or slow the instrument percusses during treatment. For this particular protocol, the limit 3306 is set at 60, the force 3308 is set at 3 pounds, and the frequency 3310 is set at 20 Hz. While these values are pre-set, the therapist can adjust as necessary given the suitability for a given patient.

After approving the treatment parameters, the therapist can apply the treatment head to the target spot on the shoulder in the spot 3304 as shown in FIG. 33 and start the treatment. The therapist can queue the patient to begin moving his or her body in the fashion as shown in the animation. The system automatically shuts off after the treatment is complete (e.g., after the device has percussed the number of times defined by the limit 3306). Of note, there is no pre-test and post-test as the treatment parameters are determined by clinical study given the particular point of treatment and the motion of the patient.

The therapy continues for each of the three treatment points as illustrated in FIG. 32. The shoulder internal rotation may use a frequency of 18 Hz, a force of 3 lbs., and a limit of 60 impacts. The therapy continues for each of the two treatment points as illustrated in FIG. 32. The shoulder external rotation may use a frequency of 17 Hz, a force of 3 lbs., and a limit of 60 impacts. The therapy continues for each of the three treatment points as illustrated in FIG. 32. The shoulder abduction may use a frequency of 16 Hz, a force of 3 lbs., and a limit of 60 impacts. The therapy is for the target spot as illustrated in FIG. 32.

Turning back to FIG. 31, the therapist may select any of the other icons corresponding to a part of the body in which treatment is desired. The menu screen of FIG. 31 may include the treatment protocols for: the Upper Extremity; Lower Extremity; Spine, Ribs, and Pelvis; and General Health. Within the Upper Extremity, the menu includes selectable icons for the shoulder 3100, the elbow 3102, the wrist 3104, and the hand 3106. Within the Lower Extremity, the menu includes selectable icons for the hips 3108, the knee 3110, the ankle 3112, the feet 3114. Within the Spine, Ribs, and Pelvis, the menu includes selectable icons for the neck 3116, the lower back 3118, the pelvis 3120, and the ribs 3122. Within the General Health, the menu includes selectable icons associated with balance 3124, performance 3126, golf 3128, and freedom 3130.

While not illustrated for every icon, each of the menu icons of FIG. 31, when selected, will display a treatment protocol selection screen, similar to FIG. 32, where the therapist may select from a plurality of treatment protocols associated with the selected part of the body. Once a specific treatment protocol is selected, a treatment protocol navigation screen, similar to FIG. 33, appears. The specific parameters for the treatment protocol is pre-set. However, the parameters may be adjusted as necessary. An animation of patient movement is shown. This movement is shown as instruction for the patient to perform the same movement during the treatment.

The specific treatment protocols associated with each of the menu icons from FIG. 31 will be described via the following tables. It is to be appreciated that each of the protocols from the Tables (i.e., each row) is a treatment protocol, which includes a screen similar to FIG. 33. The animated motion is depicted by a representative patient performing the motion identified in the “Motion” column of the respective Table. This motion is the same motion that the patient is to perform during treatment. The treatment is to be performed according to pre-set parameters given in the Tables at the pre-set points on the patient's body, given by the location of the Points in the Tables.

Table 1 lists the protocol, limit, force, frequency, motion to be performed, and points on the body subject to treatment for the various shoulder treatment protocols when the shoulder icon 3100 is selected.

TABLE 1
Shoulder Protocol

| Protocol | Limit | Force | Freq. | Motion | Point 1 | Point 2 | Point 3 |
|---|---|---|---|---|---|---|---|
| Shoulder | 60 | 3 | 20 | Arm Circle | Anterior | Lateral | Posterior |
| Rotation 1 | | | | at 90 deg. | Deltoid | Deltoid | Deltoid |
| | | | | abduction | | | |
| Shoulder | 60 | 3 | 18 | External | Superspinatus | Posterior | Lateral |
| Rotation 2 | | | | to Internal | | Deltoid | Deltoid |
| | | | | Rotation | | | |
| Shoulder | 60 | 3 | 17 | Internal to | Superspinatus | Infraspinatus | Posterior |
| Rotation 3 | | | | External | | | Deltoid |
| | | | | Rotation | | | |
| Shoulder | 60 | 3 | 16 | Neutral to | Superior | Anterior | Lateral |
| Abduction | | | | Abduction | Genohumeral | Deltoid | Deltoid |
| | | | | | joint | | |

Table 2 lists the protocol, limit, force, frequency, motion to be performed, and points on the body subject to treatment for the various elbow treatment protocols when the elbow icon 3102 is selected.

TABLE 2
Elbow Protocol

| Protocol | Limit | Force | Freq. | Motion | Point 1 | Point 2 | Point 3 |
|---|---|---|---|---|---|---|---|
| Elbow | 40 | 3 | 24 | Extension | Biceps | Triceps | Triceps |
| Flexion | | | | to Flexion | Insertion | Muscle | Insertion |
| Elbow | 60 | 3 | 20 | Flexion | Biceps | Triceps | Triceps |
| Extension | | | | to Extension | Insertion | Insertion | Muscle |
| Elbow | 60 | 3 | 18 | Pronation | Superior | Superior | Inferior |
| Palm Up | | | | to Supination | to Radial | to Lateral | to Radial |
| | | | | | Head | Epicondyle | Head |
| Golfer's | 60 | 3 | 22 | Supination | Superior | Inferior to | Inferior |
| Elbow | | | | to Pronation | to Medial | Medial | to P2 |
| | | | | | Epicondyle | Epicondyle | |

Table 3 lists the protocol, limit, force, frequency, motion to be performed, and points on the body subject to treatment for the various wrist treatment protocols when the wrist icon 3104 is selected.

TABLE 3
Wrist Protocol

| Protocol | Limit | Force | Freq. | Motion | Point 1 | Point 2 | Point 3 |
|---|---|---|---|---|---|---|---|
| Carpal | 40 | 3 | 24 | Neutral to | Distal Ulna | Superior | Distal |
| Tunnel | | | | Flexion | | to P1 | Radius |
| Flexion | 40 | 3 | 24 | Neutral to | Inferior | Between | Inferior |
| | | | | Extension | to Radius | P1 and P3 | to Ulna |
| Medial | 60 | 3 | 20 | Neutral to | Inferior | Between | Inferior |
| Flexion | | | | Medial | to Ulna | P1 and P3 | to Radius |
| | | | | Flexion | | | |
| Rotation | 60 | 3 | 18 | Wrist | Inferior | Between | Inferior |
| | | | | Circles | to Ulna | P1 and P3 | to Radius |

Table 4 lists the protocol, limit, force, frequency, motion to be performed, and points on the body subject to treatment for the various hand treatment protocols when the hand icon 3106 is selected.

TABLE 4
Hand Protocol

| Protocol | Limit | Force | Freq. | Motion | Point 1 | Point 2 | Point 3 |
|---|---|---|---|---|---|---|---|
| Hand | 40 | 3 | 24 | Fist to | Inferior | Between P1 | Inferior |
| Closed | | | | Open | to Ulna | and P3 | to Radius |
| Hand | 40 | 3 | 24 | Open to | Inferior | Between P1 | Inferior |
| Flexion | | | | Fist | to Ulna | and P3 | to Radius |
| Hand | 60 | 3 | 20 | Fist to | Inferior | Between P1 | Inferior |
| Open | | | | Open | to Ulna | and P3 | to Radius |
| Hand | 60 | 3 | 20 | Wrist | Inferior | Between P1 | Inferior |
| Rotation | | | | Circles | to Ulna | and P3 | to Radius |

Table 5 lists the protocol, limit, force, frequency, motion to be performed, and points on the body subject to treatment for the various hip treatment protocols when the hip icon 3108 is selected.

TABLE 5
Hip Protocol

| Protocol | Limit | Force | Freq. | Motion | Point 1 | Point 2 | Point 3 |
|---|---|---|---|---|---|---|---|
| Hamstring | 50 | 4 | 20 | Neutral to | Hamstring | Inferior | ¾ down |
| | | | | Forward | Origin on | to P1 | Hamstring |
| | | | | Flexion | Ischium | | |
| Piriformis | 50 | 4 | 20 | Neutral to | Lateral to S3 | Center of | Posterior |
| | | | | Hip | on Gluteus | Gluteus | Femoral |
| | | | | Flexion | Maximus | Maximus | Head |
| Sciatica | 50 | 4 | 20 | Neutral to | Lateral to S3 | Center of | Mid |
| | | | | Forward | on Gluteus | Gluteus | Hamstring |
| | | | | Flexion | Maximus | Maximus | |
| TFL | 60 | 3 | 22 | Neutral to | IT Band | Inferior | Inferior |
| Stretch | | | | Abduction | Inferior to | to P1 | to P2 |
| | | | | | Femoral Head | | |

Table 6 lists the protocol, limit, force, frequency, motion to be performed, and points on the body subject to treatment for the various knee treatment protocols when the knee icon 3110 is selected.

TABLE 6
Knee Protocol

| Protocol | Limit | Force | Freq. | Motion | Points |
|---|---|---|---|---|---|
| Extension | 45 | 3 | 24 | Flexion to | 1 Distal Quad; 2 Patella Tendon; 3 |
| | | | | Extension | Vastus Medialus; 4 Medial Collateral |
| | | | | | Ligament; 5 Patella Ligament; 6 Tibial |
| | | | | | Tuberosity |
| External | 45 | 3 | 22 | Flexion to | 1 Distal Quad; 2 Patella Tendon; 3 |
| Rotation | | | | Extension | Lateral Collateral Ligament; 4 Medial |
| | | | | | Collateral Ligament; 5 Lateral Tibial |
| | | | | | Condyle; 6 Medial Tibial Condyle; 7 |
| | | | | | Patella Ligament |
| Flexion | 45 | 3 | 20 | Tibial | 1 Distal Quad; 2 Patella Tendon; 3 |
| | | | | External | Medial Collateral Ligament; 4 Medial |
| | | | | Rotation | Hamstring Insertion; 5 Patella Ligament; |
| | | | | | 6 Tibial Tuberosity |
| Neutral | 30 | 4 | 24 | Gentle | 1 Patella Tendon; 2 Medial Collateral |
| | | | | Swinging | Ligament; 3 Patella Ligament |
| | | | | Motion | |

Table 7 lists the protocol, limit, force, frequency, motion to be performed, and points on the body subject to treatment for the various ankle treatment protocols when the ankle icon 3112 is selected.

TABLE 7
Ankle Protocol

| Protocol | Limit | Force | Freq. | Motion | Points |
|---|---|---|---|---|---|
| Roll In | 60 | 3 | 20 | Inversion | 1 Talus; 2 Cuneiforms; 3 Cuboid |
| Roil Out | 60 | 3 | 22 | Eversion | 1 Talus; 2 Cuneiforms; 3 Cuboid |
| Achilles | 45 | 4 | 24 | Ankle | 1 Achilles Below Gastrocnemius; 2 |
| Tendon | | | | Dorsiflexion | Inferior to 1; 3 Inferior to 2; 4 Achilles |
| | | | | | Insertion |
| Cuboid | 60 | 4 | 20 | Toe | 1 Cuboid/5^{th} Metatarsal Head; 2 |
| | | | | Extension | Cuboid/Lateral Cuneiform; 3 |
| | | | | | Cuboid/Calcaneus |

Table 8 lists the protocol, limit, force, frequency, motion to be performed, and points on the body subject to treatment for the various foot treatment protocols when the foot icon 3114 is selected.

TABLE 8
Foot Protocol

| Protocol | Limit | Force | Freq. | Motion | Point 1 | Point 2 | Point 3 |
|---|---|---|---|---|---|---|---|
| Foot | 50 | 3 | 22 | Plantarflexion | Distal 1^{st} | Cuneiform | Cuboid |
| Extension | | | | | Metatarsal | | |
| | | | | | Head | | |
| Foot | 50 | 3 | 22 | Dorsiflexion | Medial | Navicular | Proximal |
| Flexion | | | | | Cuneiform | | 1^{st} |
| | | | | | | | Metatarsal |
| | | | | | | | Head |
| Foot Roll | 40 | 3 | 24 | Inversion | Cuboid | Talus | Cuneiforms |
| In | | | | | | | |
| Foot Roll | 40 | 3 | 24 | Eversion | Cuboid | Talus | Cuneiforms |
| Out | | | | | | | |

Table 9 lists the protocol, limit, force, frequency, motion to be performed, and points on the body subject to treatment for the various neck treatment protocols when the neck icon 3116 is selected.

TABLE 9
Neck Protocol

| Protocol | Limit | Force | Free. | Motion | Point 1 | Point 2 | Point 3 |
|---|---|---|---|---|---|---|---|
| Mid | 50 | 3 | 18 | Neutral to | Paracervical | Paracervical | Paracervical |
| Cervical | | | | Lateral | Musculature | Musculature | Musculature |
| ROM | | | | Flexion | Lateral to C5 | Lateral to C6 | lateral to C7 |
| Neck | 60 | 3 | 16 | Neutral to | SCM Origin | Paracervical | Paracervical |
| Flexion | | | | Flexion | | Musculature | Musculature |
| | | | | | | Lateral to C3 | Lateral to C5 |
| Neck | 60 | 2 | 18 | Neutral to | Paracervical | Paracervical | Paracervical |
| Chin Out | | | | Protraction | Musculature | Musculature | Musculature |
| | | | | | Lateral to C5 | Lateral to C6 | lateral to C7 |
| Neck | 60 | 2 | 18 | Neutral to | Paracervical | Paracervical | Paracervical |
| Chin In | | | | Retraction | Musculature | Musculature | Musculature |
| | | | | | Lateral to C5 | Lateral to C6 | lateral to C7 |

Table 10 lists the protocol, limit, force, frequency, motion to be performed, and points on the body subject to treatment for the various lower back treatment protocols when the lower back icon 3118 is selected.

TABLE 10
Lower Back Protocol

| Protocol | Limit | Force | Freq. | Motion | Point 1 | Point 2 | Point 3 |
|---|---|---|---|---|---|---|---|
| Low | 40 | 4 | 24 | Neutral to | Paralumbar | Paralumbar | Paralumbar |
| Back | | | | Low back | Musculature | Musculature | Musculature |
| Lumbar | | | | Flexion | Lateral to L1 | Lateral to L3 | Lateral to L5 |
| Low | 40 | 4 | 22 | Neutral to | Gluteus | Gluteus | SI Joint |
| Back SI | | | | Hip | Medius | Maximus | |
| LOC | | | | Extension | | | |
| Low | 60 | 3 | 18 | None | Anterior | Superior | Posterior |
| Back | | | | | Fibular Head | Fibular Head | Fibular |
| Fibular | | | | | | | Head |
| Head | | | | | | | |
| Reflex | | | | | | | |
| Low | 60 | 4 | 20 | Neutral to | Paralumbar | SI Joint | Gluteus |
| Back | | | | Lateral | Musculature | | Maximus |
| Sciatica | | | | Flex on | Lateral to L4 | | |
| | | | | Opposite | | | |
| | | | | side | | | |

Table 11 lists the protocol, limit, force, frequency, motion to be performed, and points on the body subject to treatment for the various pelvis treatment protocols when the pelvis icon 3120 is selected.

TABLE 11
Pelvis Protocol

| Protocol | Limit | Force | Freq. | Motion | Points |
|---|---|---|---|---|---|
| Pelvis | 60 | 3 | 16 | Neutral to | 1 Right Superior Scapular Boarder; 2 |
| Distance | | | | Trunk | Right Medial Scapular Boarder; 3 Right |
| Release 1 | | | | Rotation | Inferior Scapular Boarder; 4 Left |
| | | | | | Superior Scapular Boarder; 5 Left |
| | | | | | Medial Scapular Boarder; 6 Left Inferior |
| | | | | | Scapular Boarder |
| Pelvis | 60 | 3 | 22 | Neutral to | 1 Right Superior Scapular Boarder; 2 |
| Distance | | | | Trunk lateral | Right Medial Scapular Boarder; 3 Right |
| Release 2 | | | | Flexion | Inferior Scapular Boarder; 4 Left |
| | | | | | Superior Scapular Boarder; 5 Left |
| | | | | | Medial Scapular Boarder; 6 Left inferior |
| | | | | | Scapular Boarder |
| Pelvis | 60 | 3 | 20 | Neutral to | 1 Gluteus Medius; 2 Gluteus Maximus; |
| Relax | | | | hip Flexion | 3 SI Joint |
| Sacral | 60 | 3 | 18 | Seated with | 1 Left SI; 2 Right SI; 3 S5 |
| Balance | | | | Hip | |
| | | | | Abduction | |

Table 12 lists the protocol, limit, force, frequency, motion to be performed, and points on the body subject to treatment for the various ribs treatment protocols when the ribs icon 3122 is selected.

TABLE 12
Ribs Protocol

| Protocol | Limit | Force | Freq. | Motion | Points |
|---|---|---|---|---|---|
| Mid Back | 60 | 3 | 18 | Neutral to | 1 Parathoracic Musculature Between |
| Extension | | | | Trunk | Scapula; 2 Parathoracic Musculature |
| | | | | Extension | Between Scapula; 3 Parathoracic |
| | | | | | Musculature Between Scapula |
| Mid Back | 60 | 3 | 20 | Neutral to | 1 Parathoracic Musculature Between |
| Flexion | | | | Trunk | Scapula; 2 Parathoracic Musculature |
| | | | | Flexion | Between Scapula; 3 Parathoracic |
| | | | | | Musculature Between Scapula |
| Mid Back | 60 | 3 | 20 | Neutral to | 1 Right Superior Scapular Boarder; 2 |
| Rotation | | | | Trunk | Right Medial Scapular Boarder; 3 Right |
| | | | | Rotation | Inferior Scapular Boarder; 4 Left |
| | | | | | Superior Scapular Boarder; 5 Left |
| | | | | | Medial Scapular Boarder; 6 Left |
| | | | | | Inferior Scapular Boarder |
| Mid Back | 60 | 3 | 22 | Lateral | 1 Left Parathoracic Musculature |
| Lateral | | | | Flexion | Lateral to T6; 2 Left Parathoracic |
| Bending | | | | | Musculature Lateral to T9; 3 Left |
| | | | | | Parathoracic Musculature Lateral to |
| | | | | | T12; 4 Right Parathoracic Musculature |
| | | | | | Lateral to T6; 5 Right Parathoracic |
| | | | | | Musculature Lateral to T9; 6 Right |
| | | | | | Parathoracic Musculature Lateral to |
| | | | | | T12 |

Table 13 lists the protocol, limit, force, frequency, motion to be performed, and points on the body subject to treatment for the various balance treatment protocols when the balance icon 3124 is selected.

TABLE 13
Balance Protocol

| Protocol | Limit | Force | Freq. | Motion | Points |
|---|---|---|---|---|---|
| HP | 60 | 3 | 18 | Straight Leg | 1 Right Gluteus Maximus Lateral to |
| | | | | Lifts | S3; 2 Left Gluteus Maximus Lateral to |
| | | | | | S3; 3 Right Posterior Femoral Head; 4 |
| | | | | | Left Posterior Femoral Head; 5 Right |
| | | | | | S3; 6 Left S3 |
| Ankle | 60 | 3 | 20 | Various | 1 Left Cuboid Plantarflexion and |
| Motion | | | | | Dorsiflexion; 2 Left Cuneiform |
| | | | | | Plantarflexion and Dorsiflexion; 3 Left |
| | | | | | Cuneiform Clockwise rotation × 3; 4 |
| | | | | | Left Cuneiform Counter-Clockwise |
| | | | | | rotation × 3; Repeat for Right. |
| Leg Up | 60 | 3 | 18 | Various | 1 Left Posterior Femoral Head Hip |
| and Over | | | | | Flexion and Abduction; 2 Left Anterior |
| | | | | | Femoral Head Hip Flexion and |
| | | | | | Abduction; Repeat for Right. |
| Hip and | 45 | 3 | 22 | Various | 1 Left Posterior Fibular Head; 2 Left |
| Leg | | | | | Posterior Femoral Head; 3 Left |
| Forward | | | | | Posterior Lateral Malleolus; Repeat for |
| Stretch | | | | | Right. |

Table 14 lists the protocol, limit, force, frequency, motion to be performed, and points on the body subject to treatment for the various performance treatment protocols when the performance icon 3126 is selected.

TABLE 14
Performance Protocol

| Protocol | Limit | Force | Freq. | Motion | Points |
|---|---|---|---|---|---|
| Core | 60 | 3 | 18 | Shoulder | 1 Right of L5; 2 Right of L1; 3 Right of |
| Motion | | | | Flexion then | T8; Repeat for Left. |
| | | | | Horizontal | |
| | | | | Adduction | |
| | | | | then Trunk | |
| | | | | Rotation | |
| Athletic | 60 | 3 | 18 | Various | 1 Right Gluteus Maximus lateral to S3 |
| Balance | | | | | with Hip Flexion; 2 Right Posterior |
| | | | | | Femoral Head with Hip Flexion; 3 |
| | | | | | Right Anterior Femoral head with Hip |
| | | | | | Flexion; Repeat for Left. |
| Look Both | 50 | 3 | 20 | Various | 1 Left Proximal Trapezius with Right |
| Ways | | | | | Rotation; 2 Right Proximal Trapezius |
| | | | | | with Left Rotation; 3 Left Distal |
| | | | | | Trapezius with Right Rotation; 4 Right |
| | | | | | Distal Trapezius with Left Rotation |
| Tae Kwon | 50 | 3 | 20 | Various, | 1 Left of T4 on Parathoracic |
| Do | | | | including | Musculature; 2 Left of T8 on |
| | | | | punches | Parathoracic Musculature; 3 Left of L1 |
| | | | | | on Paralumbar Musculature; 4 Left of |
| | | | | | L5 on Paralumbar Musculature; repeat |
| | | | | | for Right. |

Table 15 lists the protocol, limit, force, frequency, motion to be performed, and points on the body subject to treatment for the various golf treatment protocols when the golf icon 3126 is selected.

TABLE 15
Golf Protocol

| Protocol | Limit | Force | Freq. | Motion | Points |
|---|---|---|---|---|---|
| Golf 1 | 60 | 3 | 18 | Right | 1 Left Posterior Femoral Head; 2 Left |
| | | | | Rotation | Lateral to L4 on Paralumbar |
| | | | | (swing), | Musculature; 3 Left Lateral to T10 on |
| | | | | then left | Parathoracic Musculature; repeat for |
| | | | | | Right side during left rotation. |
| Golf 2 | 60 | 3 | 20 | Various | 1 Lateral to T12 on Right Parathoracic |
| | | | | | Musculature with L Trunk Rotation; |
| | | | | | repeat for left with right trunk rotation; |
| | | | | | Lateral to T8 on Left Parathoracic |
| | | | | | Musculature with Left Trunk Rotation; |
| | | | | | repeat for Right with Right Rotation |
| Golf 3 | 50 | 3 | 22 | Various | 1 Right Gluteus Maximus Lateral to S5 |
| | | | | | with Right Trunk Rotation; 2 Right |
| | | | | | Posterior Femoral Head with Right |
| | | | | | Trunk Rotation; repeat for Left with left |
| | | | | | rotation |
| Golf 4 | 45 | 3 | 24 | Various | 1 Right Gluteus Maximus Lateral to S5 |
| | | | | | with Right Hip Flexion/Abduction then |
| | | | | | Hip Flexion/Adduction; 2 Right Medial |
| | | | | | Gluteus Maximus with Right Hip |
| | | | | | Flexion/Abduction then Hip |
| | | | | | Flexion/Adduction; 3 Right Posterior |
| | | | | | Femoral Head with Right Hip |
| | | | | | Flexion/Abduction then Hip |
| | | | | | Flexion/Adduction; repeat for Left. |

Table 16 lists the protocol, limit, force, frequency, motion to be performed, and points on the body subject to treatment for the various freedom treatment protocols when the freedom icon 3126 is selected.

TABLE 16
Freedom Protocol

| Protocol | Limit | Force | Freq. | Motion | Points |
|---|---|---|---|---|---|
| Arm | 50 | 3 | 22 | Raise Arm | 1 Right Infraspinatus with Shoulder |
| Motion | | | | | Forward Flexion; 2 Right |
| | | | | | Supraspinatus with Shoulder Forward |
| | | | | | Flexion; 3 Right Infraspinatus with |
| | | | | | Shoulder Forward Flexion; 4 Right |
| | | | | | Supraspinatus with Shoulder Forward |
| | | | | | Flexion; repeat for Left. |
| Knee Kick | 45 | 3 | 20 | Hip | 1 Right Patella Tendon; 2 Right Lateral |
| | | | | Flexion/knee | Collateral Ligament; 3 Right Patella |
| | | | | extension | Ligament; repeat for left. |
| | | | | then knee | |
| | | | | flexion/hip | |
| | | | | extension | |
| Swimming | 50 | 3 | 22 | Various | 1 Right Rhomboid with Shoulder |
| | | | | including | Forward Flexion; 2 repeat for Left; 3 |
| | | | | swimming | Repeat entire operation as necessary. |
| | | | | strokes | |
| Mid Back | 60 | 3 | 20 | Sitting upper | 1 Lateral to T10 Parathoracic |
| Flexibility | | | | body twist | Musculature on Left with Right Trunk |
| | | | | | Rotation; 2 Lateral to T8 Parathoracic |
| | | | | | Musculature on Left with Right Trunk |
| | | | | | Rotation; 3 Left Rhomboid with Left |
| | | | | | Trunk Rotation; repeat on Right side |
| | | | | | with opposite trunk rotation. |

This treatment protocol is not static, but is dynamic. This is the reason for referring the therapy of the soft tissue as the mechanical motion therapy. This treatment is different from the adjustment of the joints or spines. The joints are adjusted in a stationary position, whereas the soft tissue is percussed with the patient in motion. The soft tissue includes the muscles, the tendons, the ligaments, the mechanoreceptors, and the proprioceptors, among others. The frequency or speed of the hit or tap from the treatment head for the soft tissue is higher than that for the joint or spine.

It will be appreciated by those skilled in the art that the number of icons for the soft tissue on the home screen may vary and also the number of protocols may vary.

In some variations, a patient may get the therapy with the system for treating soft tissue, but may not get an adjustment with the system for treating joint mobility. In some variations, a patient may get an adjustment with the system for treating joint mobility, but may not get the therapy for the soft tissue.

Any ranges cited herein are inclusive. The terms “substantially” and “about” used throughout this specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the method and system, which, as a matter of language, might be said to fall there between.

What is claimed is:

1. A system for treating a joint of a patient, the system comprising:
a treatment head comprising a probe tip, a pressure sensor, and a wave sensor, the treatment head configured to mechanically oscillate the probe tip so as to provide treatment;
a computing device in communication with the treatment head and comprising a processing device and a computer-readable medium with one or more executable instructions stored thereon, wherein the processing device of the computing device executes the one or more instructions to perform the operations of:
receiving, from the wave sensor, a first pre-test waveform representing a first factor of joint mobility at a first location on the patient;
determining a first treatment protocol for use at the first location based on the first factor of joint mobility at the first location;
signaling the treatment head to mechanically oscillate the probe tip at the first location according to the first treatment protocol including a force, amplitude, and cycles;
receiving, from the wave sensor, a first post-test waveform representing a second factor of joint mobility at the first location on the patient; and
determining a first measure of joint mobility based on a comparison of the first and second factors of joint mobility.

2. The system of claim 1, wherein the operations further include:
receiving, from the wave sensor, a second pre-test waveform representing a third factor of joint mobility at a second location on the patient;
determining a second treatment protocol for use at the second location based on the third factor of joint mobility at the second location;
signaling the treatment head to mechanically oscillate the probe tip at the second location according to the second treatment protocol including a force, amplitude, and cycles;
receiving, from the wave sensor, a second post-test waveform representing a fourth factor of joint mobility at the second location on the patient; and
determining a second measure of joint mobility based on a comparison of the third and fourth factors of joint mobility.

3. The system of claim 2, wherein the first and second locations are on opposite sides of the joint.

4. The system of claim 3, wherein the first and second locations are on a posterior side of the joint and an anterior side of the joint, respectively.

5. The system of claim 3, wherein the first and second locations are on a superior side of the joint and an inferior side of the joint, respectively.

6. The system of claim 2, wherein the operations further include: calculating a joint function index based on the first and second measures of joint mobility.

7. The system of claim 6, wherein calculating the joint function index includes a calculation of the differences between the first pre-test waveform and the first post-test waveform and the difference between the second pre-test waveform and the second post-test waveform.

8. The system of claim 6, wherein calculating the joint function index includes a summation of: a first difference between the first pre-test waveform and the first post-test waveform; and a second difference between the second pre-test waveform and the second post-test waveform.

9. The system of claim 1, wherein the treatment head includes an electrode operably coupled to the probe, the electrode being configured to deliver an electrical stimulation to the patient.

10. A system for treating a joint of a patient, the system comprising:
a treatment head including a probe, a force impulse wave sensor, and a pressure sensor, the pressure sensor configured so that when the probe is pressed against the joint and reaches a predetermined pressure, the pressure sensor causes a release of current such that the probe delivers a mechanical force impulse to the joint, and the force impulse wave sensor configured to sense a frequency of the mechanical force impulse associated with at least one treatment point of the joint;
a computer portion including a central processing unit (CPU) in communication with the treatment head; and
a first display screen in communication with the computer portion, the first display screen configured to: display a plurality of target spots on the joint for pre-test, treatment, and post-test; and to display the pre-test and post test results of each of the plurality of target spots,
wherein the treatment head is configured to perform the pre-test, the treatment, and the post-test in succession for a first target spot of the plurality of target spots before performing treatment to a second target spot of the plurality of target spots,
wherein the computer portion is configured to perform the pretest and post-test analysis associated with the plurality of target points to evaluate improvement of the joint after the treatment, wherein, for each of the plurality of target spots, at least one treatment protocol associated with the treatment is determined based on the frequency sensed by the force impulse wave sensor in response to the mechanical force impulse from the pre-test.

11. The system of claim 10, wherein the treatment comprises a number of impulses.

12. The system of claim 10, wherein the treatment comprises a frequency of impulses.

13. The system of claim 10, wherein the joint comprises one of shoulder, elbow, wrist, hip, knee, or ankle.

14. The system of claim 10, wherein the frequency is within a range of 0.1 Hz to 12 Hz.

15. The system of claim 10, further comprising an electrical stimulation device in communication with the computer portion.

16. The system of claim 15, wherein the treatment protocol comprises electrical stimulation via the electrical stimulation device.

17. The system of claim 10, further comprising a second display screen in communication with the computer portion, the second display screen configured to display a highlighted target area for treatment on a live human model and to display an application of the treatment head on the highlighted treatment point of the live human model.

18. The system of claim 10, wherein the computer portion is configured to calculate a joint function index based upon the pre-tests and the post-tests on the plurality of target spots, the joint function index indicating a state of joint mobility of the joint following treatment.

19. The system of claim 18, wherein a computation of the joint function index includes differences between waveforms of the pre-test and post-test associated with the treatment of each of the plurality of target spots.

20. The system of claim 18, wherein a computation of the joint function index includes a summation of the differences between waveforms of the pre-test and post-test associated with the treatment of each of the plurality of target spots.

21. A system for treating soft tissue of a patient, the system comprising:
a treatment head including a probe, and a pressure sensor configured so that when the probe is pressed against the soft tissue and reaches a predetermined pressure, the pressure sensor causes a release of current such that the probe delivers a mechanical force impulse to the soft tissue;
a computer portion including a central processing unit (CPU) in communication with a memory device and the treatment head, the memory device having a plurality of treatment protocols for each of a plurality of treatment points on the soft tissue stored thereon; and
at least one display screen in communication with the computer portion, the at least one display screen configured to: display the plurality of treatment points on a representation of the patient; and display the representation of the patient performing a movement of a body part to be replicated by the patient during the treatment of the soft tissue;
wherein the treatment head is configured to perform the plurality of treatment protocols for each of a plurality of treatment points on the soft tissue.

22. The system of claim 21, wherein the soft tissue comprises one of the plurality of treatment points comprising shoulder, elbow, wrist, hand, hips, knee, ankle, feet, neck, lower back, middle back, pelvis, ribs, arm, and leg.

23. The system of claim 22, wherein the treatment head is configured to provide oscillating force impulses at a frequency ranging from 12 Hz to 30 Hz.

24. The system of claim 21, wherein the treatment head is configured to treat the soft tissue to create one or more neuro-pathways in the soft tissue.

25. The system of claim 21, further comprising an electrode operably coupled to the probe to deliver an electrical stimulation,

26. A method of treating joint of a patient, the method comprising:
a) contacting the patient at a first location of a plurality of locations at least partially surrounding the joint with a probe tip of a treatment head of a treatment system, the treatment head comprising a probe having the probe tip, a pressure sensor, and a wave sensor, the treatment head configured to mechanically oscillate the probe tip so as to provide treatment, the treatment head in communication with a computer of the treatment system, the computer comprising a memory and a central processing unit (CPU), the computer in communication with a display device of the treatment system;
b) applying a first compressive force with the probe tip to the first location proximate the joint, thereby the wave sensor receives a pre-test waveform representing a first factor of joint mobility at the first location, the CPU determining a first treatment protocol for use at the first location based on the first factor of joint mobility at the first location;
c) applying a second compressive force with the probe tip at the first location, the second compressive force causing the probe tip of the treatment head to deliver percussive impacts according to the first treatment protocol including a force, amplitude, and cycles; and
d) applying a third compressive force with the probe tip at the first location, thereby the wave sensor receives a post-test waveform representing a second factor of joint mobility at the first location, the CPU determining a first measure of joint mobility based on a comparison of the first and second factors of joint mobility.

27. The method of claim 26, further comprising:
repeating the steps of (a)-(d) to each of a second location, a third location, a fourth location, and a fifth location.

28. The method of claim 27, wherein the first location is on a posterior side of the joint, the second location is on an anterior side of the joint, the third location is on a lateral side of the joint, the fourth location is on a superior side of the joint, and the fifth location is on an inferior side of the joint.

29. The method of claim 27, wherein the computer portion calculates a joint function index based on the pre-tests of the five locations.

30. The method of claim 26, wherein the percussive impacts are delivered to the first location.

31. The method of claim 30, wherein the percussive impacts and an electrical stimulation delivered to the first location are delivered generally simultaneously, wherein the electrical stimulation is created from an electrode operably coupled to the probe.

32. The method of claim 26, wherein the joint comprises one of shoulder, elbow, wrist, hip, knee, or ankle.

33. A method of treating spine of a patient, the method comprising:
a) contacting the patient at a first location of a plurality of locations at least partially surrounding the spine with a probe tip of a treatment head of a treatment system, the treatment head comprising a probe having the probe tip, a pressure sensor, and a wave sensor, the treatment head configured to mechanically oscillate the probe tip so as to provide treatment, the treatment head in communication with a computer of the treatment system, the computer comprising a memory and a central processing unit (CPU), the computer in communication with a display device of the treatment system;
b) applying a first compressive force with the probe tip to the first location proximate the spine;
c) repeating the steps of a) and b) for each of the remaining plurality of locations, thereby the wave sensor receives a pre-test waveform representing a first factor of spine mobility at the plurality of locations,
d) selecting a treatment location from the plurality of locations, the CPU determining a treatment protocol for use at the treatment location based on the first factor of spine mobility at the treatment location and at least one of the first factor of spine mobility at one or more of the plurality of locations;
e) applying a second compressive force with the probe tip at the treatment location, the second compressive force causing the probe tip of the treatment head to deliver percussive impacts according to the treatment protocol including a force, amplitude, and cycles; and
f) applying a third compressive force with the probe tip at the plurality of locations, thereby the wave sensor receives a post-test waveform representing a second factor of spine mobility at the plurality of locations, the CPU determining a measure of spine mobility based on a comparison of the first and second factors of spine mobility.

34. The method of claim 33, wherein the percussive impacts are delivered to the treatment location.

35. The method of claim 33, wherein the spine comprises one of cervical spine portion, thoracic spine portion, lumbar spine portion, or sacral spine portion.

36. The method of claim 33, wherein each of cervical spine portion, thoracic spine portion, lumbar spine portion, and sacral spine portion comprises a respective plurality of treatment points.
